(19) United States
(12) Patent Application Publication
(10) Pub. No.: US 2025/0228455 A1
(43) Pub. Date: Jul. 17, 2025

(54) METHODS AND APPARATUS FOR DETECTING OCULAR DISEASES

(71) Applicant: EyeCare SpA, Las Condes (CL)

(72) Inventors: Maria Eliana MANQUEZ HATTA, Santiago (CL); Mauricio Castro, Santiago (CL); Pablo Riedemann, Santiago (CL); Jose Vines Lopez, Santiago (CL)

(73) Assignee: EyeCare SpA, Las Condes (CL)

(21) Appl. No.: 19/170,631

(22) Filed: Apr. 4, 2025

Related U.S. Application Data
(63) Continuation of application No. PCT/IB2023/060027, filed on Oct. 5, 2023.
(60) Provisional application No. 63/519,762, filed on Aug. 15, 2023.
(60) Provisional application No. 63/413,603, filed on Oct. 5, 2022.

(51) Int. Cl. A61B 3/14; A61B 3/00; H04N 23/611; H04N 23/63; H04N 23/67; H04N 23/69; H04N 23/74

(52) U.S. Cl. CPC A61B 3/145; A61B 3/0041; H04N 23/611; H04N 23/632; H04N 23/67; H04N 23/74; A61B 2503/04; A61B 2503/06; H04N 23/69

(57) ABSTRACT

A system for diagnosing ocular diseases of a subject includes a mobile device (e.g., a smart phone, a tablet) to facilitate acquisition of imagery of the subject's eyes and a backend server to evaluate the imagery using a machine learning model to determine if the subject's eyes are health or unhealthy. The mobile device may execute an application to facilitate the acquisition of imagery for evaluation. In one aspect, the application may gradually ramp up the intensity of a flash when capturing imagery of the subject to reduce or, in some instances, mitigate delays caused by the camera adjusting its white balance. In another aspect, an external flash device may be coupled to the mobile device to provide a light source that is more powerful and more responsive than conventional flash devices.

### CROSS-REFERENCE TO RELATED APPLICATION(S)

The present application is a bypass continuation of International Application No. PCT/IB2023/060027, filed Oct. 5, 2023, entitled “METHODS AND APPARATUS FOR DETECTING OCULAR DISEASES,” claims priority to U.S. Provisional Application No. 63/413,603, filed Oct. 5, 2022, entitled “METHODS AND APPARATUS FOR DETECTING OCULAR DISEASES,” and U.S. Provisional Application No. 63/519,762, filed Aug. 15, 2023, entitled “METHODS AND APPARATUS FOR DETECTION OF OPTICAL DISEASES.” Each of the aforementioned applications is incorporated by reference herein in its entirety.

### BACKGROUND

An estimate based on the National Health Survey (ENS 2007) indicates that at least 1.5% to 2.6% of the Chilean population has some visual impairment, of this percentage is estimated that at least ¼ of them has chronic defects classified as blindness. The world situation is not so different, and this reveals that there are at least 12 million children under the age of 10, which is the age group of preventive control, that suffer from visual impairment due to refractive error (myopia, strabismus or astigmatism). In addition, there are more severe cases like ocular cancer that affects 1 in 12,000 live births, which is usually seen in children up to 5 years old. All of these conditions and others, in most cases, may be corrected without major complications with a preventive diagnosis and effective treatment in infants from birth to about 5 years old, preventing these disorders getting worse with time and treatment being too expensive, ineffective or simply being too late to be implemented.

The red pupillary reflex is fairly well understood by ophthalmologists and pediatric specialists worldwide and has been used as a diagnostic instrument around the world since the 1960s. Normally, the light reaches the retina and a portion of it is reflected off the pupil by the choroid or posterior uvea, which is a layer of small vessels and pigmented cells located near the retina. The reflected light, seen from a coaxial instrument to the optical plane of the eye, normally present a reddish color, due to the color of blood and pigments of the cells, so this color can vary from shiny reddish or yellowish in people with light pigmentation to a more grayish red or dark pigmentation in people with dark pigmentation. In 1962, Bruckner (Bruckner R. Exakte Strabismus diagnostic bei ½-3 jahrigen Kindern mit einem einfachen Verfahren, dem “Durchleuchtungstest.” Ophthalmologica 1962; 144:184-98) described abnormalities in the pupillary reflex as well as in quality, intensity, symmetry or presence of abnormal figures, therefore, pupilar red color test is also known as the Bruckner test. Another similar test is the Hirschberg test, which uses the corneal reflex to detect misalignment of the eyes, which enables to diagnose some degree of strabismus (Wheeler, M. “Objective Strabismometry in Young Children.” Trans Am Ophthalmol Soc 1942; 40: 547-564). In summary, these tests are used to detect misalignment of the eyes (strabismus), different sizes of the eyes (anisometropy), abnormal growths in the eye (tumors), opacity (cataract) and any abnormalities in the light refraction (myopia, hyperopia, astigmatism).

The evaluation of the pupillary and corneal reflexes is a medical procedure that can be performed with an ophthalmoscope, an instrument invented by Francis A. Welch and William Noah Allyn in 1915 and used since the last century. Today, his company Welch Allyn, has products that follow this line as Pan Optic™. There are also photographic screening type portable devices for the evaluation of pupilar red color as Plusoptix (Patent application No. WO9966829) or Spot™ Photoscreener (Patent Application No. EP2676441 A2), but the cost ranges between USD 100 to 500, they weigh about 1 kg, and also require experience in interpreting the observed images.

### SUMMARY

The Inventors have recognized and appreciated the detection of ocular diseases in individuals, particularly young children and infants, may be readily corrected provided the ocular diseases are detected and diagnosed early. However, the Inventors have also recognized the detection of ocular diseases typically requires continuous medical supervision and examinations, which are carried out using high-cost instruments that also require operation by trained specialists. Moreover, for the group of infants (0-5 years), there are two key problems in performing these tests: it is difficult to make that infants focus their gaze intently to any device that performs the test and also the ophthalmologist or pediatrician has only fraction of a second to capture the image before the pupil shrinks in response to the bright flash. These problems have, in some instances, led to ocular diseases in children going undetected and/or undiagnosed for prolonged periods of times (e.g., years), thus preventing pediatricians from prescribing preventive measures before the problem gets worse.

The present disclosure is thus directed to various inventive implementations of an apparatus, such as a mobile device (e.g., a smart phone, a tablet) and a system incorporating the apparatus to perform preliminary examination of a subject to facilitate rapid diagnosis of ocular disease. An executable application embodying the various inventive concepts disclosed herein may be executed by the system to facilitate, for example, acquisition of imagery of the subject's eyes. In one aspect, the various inventive improvements disclosed herein may allow for practical and reliable solutions for rapid diagnosis of ocular diseases, which allows a preliminary examination only with the use of smart phones or tablet type devices, currently used by millions of people worldwide. In another aspect, an executable application embodying the various inventive concepts disclosed herein may be run by parents, paramedics, pediatricians and ophthalmologists without the need for a more complex instrument or experience in the use of these, and effectively allows conducting a test to detect ocular diseases.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1A shows a front view of a mobile device (e.g., a smart phone).

FIG. 1B shows a rear view of the mobile device of FIG. 1A.

FIG. 2A shows an example application running on the mobile device of FIG. 1A.

FIG. 2B shows an example use of the mobile device of FIG. 2A while the subject's eyes (e.g., infant's eyes) are focused.

FIG. 3A shows a diagram of an example system architecture for diagnosing ocular diseases. The system includes the mobile device of FIG. 1A.

FIG. 3B shows a data flow diagram of an example process to acquire and process imagery of a subject's eyes using the system of FIG. 3A.

FIG. 4A shows an example subject.

FIG. 4B shows the application of FIG. 2A executing a pre-capture process where the mobile device targets a subject and shows guide messages to assist a user operating the mobile device.

FIG. 4C shows a flow chart for an example pre-capture process.

FIG. 5 shows an example capture process where a flash is used to illuminate the subject and a camera acquires imagery of the subject's red pupillary reflex.

FIG. 6 shows an example post capture process where imagery acquired by the capture process of FIG. 5 is cropped to isolate the subject's eyes.

FIG. 7 shows an example of another post capture process where an image is selected from a set of images for evaluation of ocular disease.

FIG. 8A shows an example image of a normal (i.e., healthy) red pupillary reflex.

FIG. 8B shows additional example images of normal red pupillary reflexes.

FIG. 9A shows an example image of a red pupillary reflex with a refractive error.

FIG. 9B shows another example image of a red pupillary reflex with a refractive error.

FIG. 10A shows an example image of a red pupillary reflex with Leukocoria.

FIG. 10B shows another example image of a red pupillary reflex with a tumor disease.

FIG. 11A shows an illustration of the multiple labels incorporated into a mask generated by an eye mask creator.

FIG. 11B shows an example mask from the image of FIG. 11A.

FIG. 12A shows an example image of a normal (i.e., healthy) red pupillary reflex and corresponding color and grayscale masks.

FIG. 12B shows an example image of a red pupillary reflex with a tumor disease and corresponding color and grayscale masks.

FIG. 13A shows an example image of a normal (i.e., healthy) red pupillary reflex.

FIG. 13B shows an example mask created by the eye mask creator of FIG. 11 for the image of FIG. 13A where the mask includes multiple layers to indicate the sclera, iris, and pupil of the subject's eyes.

FIG. 14 shows an example confusion matrix for the U-Net machine learning model.

FIG. 15A shows a diagram of an example external flash device.

FIG. 15B shows the external flash device of FIG. 15A coupled to a mobile device.

FIG. 15C shows an example image captured using the external flash device of FIG. 15A.

FIG. 16A shows another example external flash device with a clip.

FIG. 16B shows the external flash device of FIG. 16A coupled to a mobile device.

FIG. 17A shows another example external flash device that is adjustable along a single axis.

FIG. 17B shows the external flash device of FIG. 17A coupled to a mobile device.

FIG. 18A shows another example external flash device that is adjustable along two axes.

FIG. 18B shows the external flash device of FIG. 18A coupled to a mobile device.

### DETAILED DESCRIPTION

Following below are more detailed descriptions of various concepts related to, and implementations of, an apparatus, such as a mobile device (e.g., a smart phone, a tablet) and a system incorporating the apparatus to perform preliminary examination of a subject to facilitate rapid diagnosis of ocular disease. The inventive concepts disclosed herein may provide an accessible, easy-to-use approach to detect ocular diseases without relying upon specialized equipment and/or requiring users to be specially trained. This may be accomplished, in part, by the apparatus and the system executing one or more methods to acquire imagery of a subject's eyes, process the imagery, classify the imagery (e.g., healthy, unhealthy), and/or display on the apparatus a diagnosis of the subject's eyes (e.g., healthy, unhealthy). These foregoing methods may be executed, in part, by one or more processors in the apparatus and/or the system as part of an executable application stored in memory on the apparatus and/or the system. It should be appreciated that various concepts introduced above and discussed in greater detail below may be implemented in multiple ways. Examples of specific implementations and applications are provided primarily for illustrative purposes so as to enable those skilled in the art to practice the implementations and alternatives apparent to those skilled in the art.

The figures and example implementations described below are not meant to limit the scope of the present implementations to a single embodiment. Other implementations are possible by way of interchange of some or all of the described or illustrated elements. Moreover, where certain elements of the disclosed example implementations may be partially or fully implemented using known components, in some instances only those portions of such known components that are necessary for an understanding of the present implementations are described, and detailed descriptions of other portions of such known components are omitted so as not to obscure the present implementations.

In the discussion below, various examples of an apparatus, a system, and methods are provided, wherein a given example or set of examples showcases one or more features or aspects related to an external flash device for a mobile device, the capturing of imagery, the processing of the imagery, and the classification of the imagery. It should be appreciated that one or more features discussed in connection with a given example of an apparatus, a system, or a method may be employed in other examples of apparatuses, systems, and/or methods, respectively, according to the present disclosure, such that the various features disclosed herein may be readily combined in a given apparatus, system, or method according to the present disclosure (provided that respective features are not mutually inconsistent).

Certain dimensions and features of the apparatus and/or the system and its components and/or subsystems are described herein using the terms “approximately,” “about,” “substantially,” and/or “similar.” As used herein, the terms “approximately,” “about,” “substantially,” and/or “similar” indicates that each of the described dimensions or features is not a strict boundary or parameter and does not exclude functionally similar variations therefrom. Unless context or the description indicates otherwise, the use of the terms “approximately,” “about,” “substantially,” and/or “similar” in connection with a numerical parameter indicates that the numerical parameter includes variations that, using mathematical and industrial principles accepted in the art (e.g., rounding, measurement or other systematic errors, manufacturing tolerances, etc.), would not vary the least significant digit.

For purposes of the present discussion, the apparatuses, systems, and methods disclosed herein for detecting ocular diseases, for which various inventive improvements are disclosed herein, are sometimes referred to herein as “MDEyeCare.”

### 1. AN EXAMPLE APPARATUS AND SYSTEM FOR DIAGNOSING OCULAR DISEASES

The inventive concepts disclosed herein may be implemented in a computational application that is executed, in part, by a mobile device forming part of a system to facilitate diagnosis of ocular diseases. Herein, a mobile device may generally include, but is not limited to, a smartphone, an electronic tablet, and a laptop computer. This is accomplished, in part, by the apparatus acquiring imagery of a subject that captures the pupillary and corneal reflexes of the subject's eyes. For reference, imagery that captures a subject's pupillary reflex typically results in the subject's pupils appearing red in color if the subject's eyes are healthy.

The information obtained by capturing the pupillary and corneal reflexes of the subject can be used to evaluate the health of the subject's eyes. For example, this information may be used to perform a preliminary screening of the subject's eyes for various ocular diseases, according to the inventive concepts described below. The ocular diseases that may be detected include, but is not limited to, misalignment of the eyes (e.g., strabismus), different sizes of the eyes (e.g., anisometropy), abnormal growths in the eye (e.g., tumors), opacity of the eyes (e.g., cataract), and abnormalities in light refraction of the eyes (e.g., myopia, hyperopia, astigmatism).

In one aspect, the inventive concepts disclosed herein may be implemented using mobile devices that are readily accessible to the general population. Said another way, the inventive concepts disclosed herein do not require specialized equipment, such as a complex ophthalmic instrument, for implementation. For example, the mobile device may be any commercially available smart phone, such as an Apple iPhone, a Google Pixel, a Samsung Galaxy, and/or the like.

Unlike conventional pre-digital cameras, modern smart phones and tablets typically include a camera and a flash configured to reduce or, in most cases, eliminate the red pupillary reflex when capturing imagery. Herein, these feature are disabled and/or bypassed so that the camera and/or the flash of a mobile device is able to capture the pupillary and corneal reflexes of the subject's eyes when acquiring imagery. In other words, the mobile device used herein is configured to capture images that include, for example, red-colored pupils of a subject similar to conventional cameras. However, unlike conventional cameras, the mobile device and the system disclosed herein also executes several processes on the acquired imagery to determine whether the subject's eyes have any ocular diseases.

In another aspect, the application may readily be used by the general population without requiring any training. For example, the application may be used by parents, paramedics, pediatricians, and ophthalmologists. In this manner, the inventive concepts disclosed herein provide a way to perform early screening and detection of ocular diseases. It should be appreciated that the application disclosed herein isn't necessarily a substitute for visiting a specialist (e.g., a pediatrician, an ophthalmologist). Rather, the application may provide the user of the application and/or the subject an indication that an ocular disease may be present, which may then inform the user and/or the subject to visit a specialist to confirm or disprove the preliminary diagnosis.

In some implementations, the inventive concepts disclosed herein may be particularly suitable for performing ocular examinations of children and, in particular, infants. Infants are amongst the most vulnerable groups susceptible to ocular disease, in part, because several ocular diseases typically develop at a young age, which can often go undetected. As a result, ocular diseases that may be readily treatable early on may develop into more serious conditions in adulthood. The application does not require a child to sleep, to focus on an instrument or device for an extended period of time, or to be subjected to a long ocular examination. Additionally, the application does not require the use of any pharmacological drops to dilate the pupils of the child, which may result in undesirable side effects. Rather, the application disclosed herein may only require the ambient lighting be dimmed before imagery of the subject's eyes are captured (e.g., using a flash).

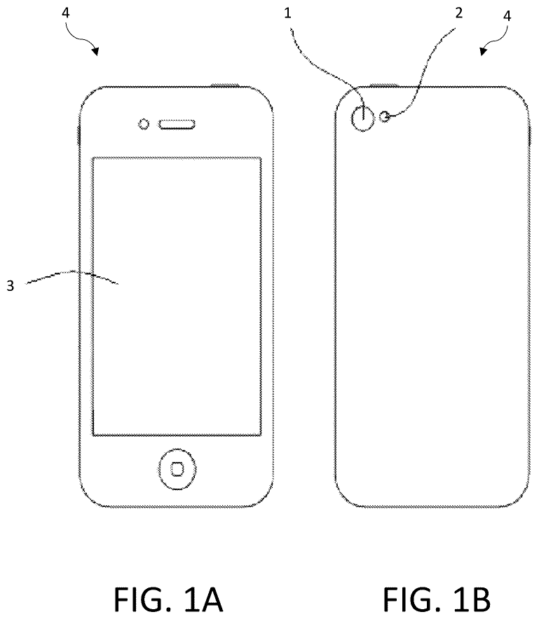

As described above, the application may be executed using various mobile devices. In one non-limiting example, FIGS. 1A and 1B show a mobile device 4 configured to execute the application disclosed herein. As shown, the mobile device 4 may be a smart phone (e.g., an Apple iPhone) with a camera 1 to acquire imagery, a flash 2 to illuminate the environment when acquiring imagery, and a display 3 (also sometimes referred to as a “screen 3”) to display information to a user of the mobile device 4 (e.g., to guide the user while acquiring imagery). The mobile device 4 may also include a communication device (e.g., an antenna to facilitate wireless communication, a port to facilitate wired communication). The mobile device 4 may be connected, for example, to the Internet through a mobile network, an Internet service provider (ISP), and/or the like. The mobile device 4 may further include one or more processors (not shown) to execute the application and memory (not shown) to store the application and imagery acquired by the mobile device 4.

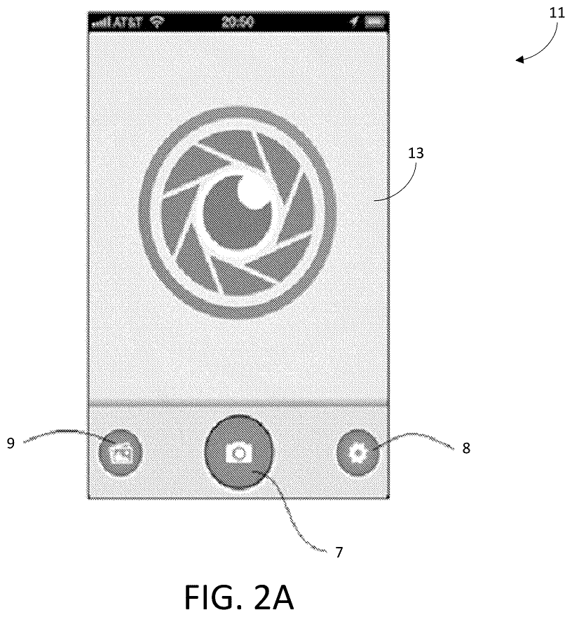

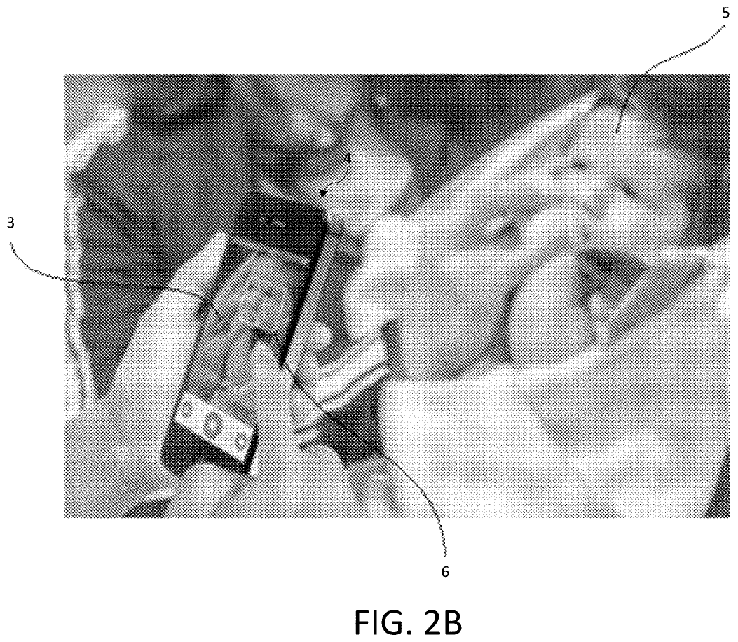

FIG. 2A shows an example graphical user interface 11 of the application displayed on the display 3 of the mobile device 4. As shown, the graphical user interface 11 may include a viewing area 13 where the subject is displayed on the display 3 based on imagery or video acquired by the camera 1 of the mobile device 4 and a button 7 to initiate one or more processes to acquire a plurality of images. The viewing area 13 may be used, for example, to assist the user of the mobile device 4 in aligning the subject's face to the camera 1 before imagery is acquired (see, for example, the pre-capture process in Section 2.1).

The graphical user interface 11 may further include a settings button 8, which when selected may provide one or more options associated with the operation of the application for the user of the mobile device 4 to change and/or turn on/off. For example, the options may include, but is not limited to, an option to log in or log out of a user account associated with the application, an option to change the displayed language used in the application, an option to adjust one or more thresholds for a brightness filter (see, for example, the post-capture processes in Section 2.3), and an option to turn on or off the brightness filter. The graphical user interface 11 also includes a view images button 9, which when selected, may allow the user of the mobile device 4 to view the imagery previously acquired by the application.

FIG. 2B shows a non-limiting example of a user using the application executed on the mobile device 4 to acquire imagery of the eyes of a subject 5 via the graphical user interface 11. As shown, the viewing area 13 of the graphical user interface 11 may display imagery acquired by the camera 1 on the display 3. The graphical user interface 11 may further provide a selection box 6 to adjust the focus of the camera 1 on the subject 5. For example, the user may touch the viewing area 13 on the display 3 to adjust the focus onto the eyes of the subject 5. Thereafter, the application may notify the user through the graphical user interface 11 to adjust the ambient lighting in the environment, e.g., by reducing or increasing the ambient lighting, so that there's sufficient lighting to detect (e.g., by the user or the application) the face of the subject 5 while allowing the pupils of the subject 5 to dilate (see, for example, Section 2.1). After adequately adjusting the ambient lighting, imagery of the subject may be acquired by selecting, for example, the button 7.

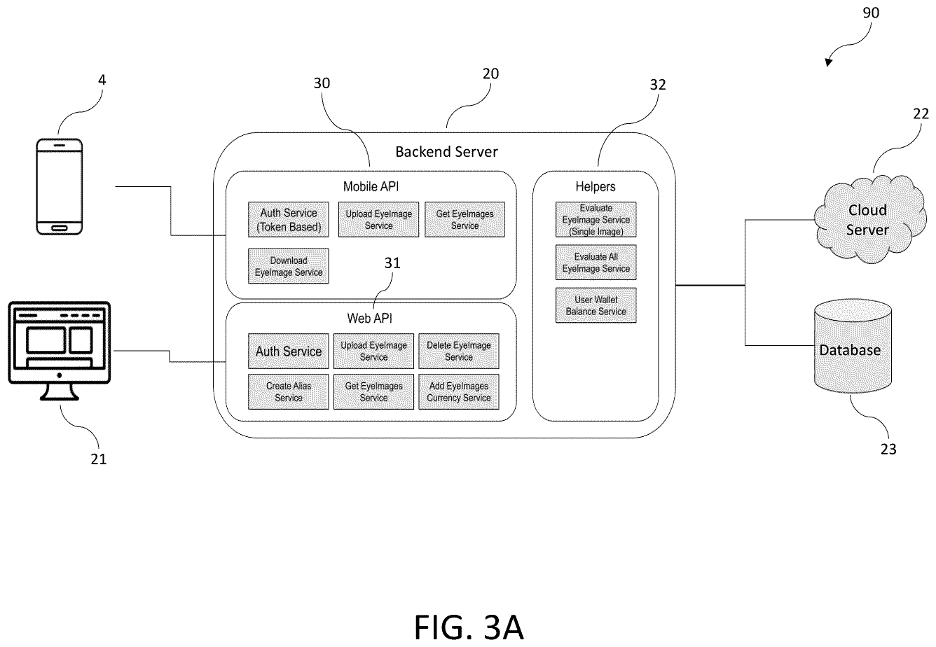

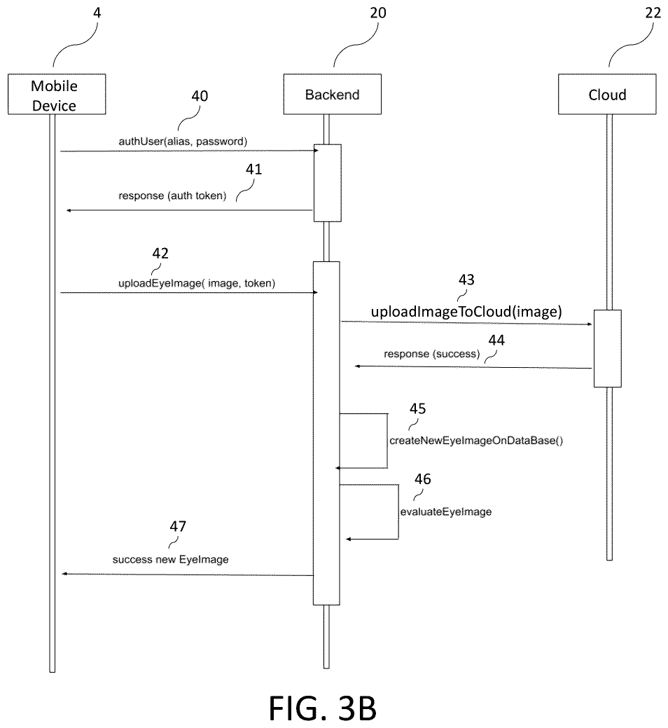

As described above, the mobile device 4 and the application may form part of a larger system that processes and evaluates imagery acquired by the mobile device 4 to assess the health of the subject's eyes. In one non-limiting example, FIG. 3A shows an example system 90, which includes the mobile device 4 described above with the application communicatively coupled to a backend server 20. The backend server 20 may facilitate communication with the mobile device 4 through use of a mobile application programming interface (API) 30. The mobile API 30 may provide several functions including, but not limited to, distribution of the application to one or more mobile devices 4, facilitating transmission of imagery from the mobile device 4 to the backend server 20 for evaluation, facilitating creation of an account associated with the user and/or subject of the mobile device 4, and authorizing access to the services provided by the system 90 for a particular user of the application (e.g., using a token-based authorization approach).

It should be appreciated the system 90 is not limited to supporting only mobile devices 4. More generally, the system 90 may allow any electronic device to use the application and/or services. For example, FIG. 3A shows a stationary device 21 (e.g., a desktop computer) may also be used to acquire imagery of a subject's eyes for evaluation of ocular disease using a web-based application. As shown, the backend server 20 may facilitate communication with the device 21 through use of a web API 31. Like the mobile API 30, the web API 31 may provide several functions including, but not limited to, providing access to a web-based application to one or more devices 21, facilitating transmission of imagery from the device 21 to the backend server 20 for evaluation, facilitating creation of an account associated with the user and/or subject of the device 21, and authorizing access to the services provided by the system 90 for a particular user of the web-based application.

In one aspect, the backend server 20 may store a machine learning model in memory trained to classify imagery of a subject's eyes according to a predetermined selection of ocular diseases. During operation, the backend server 20 may evaluate imagery from the mobile device 4 (or the stationary device 21) by passing the imagery as input to the machine learning model. The machine learning model, in turn, may provide an output indicating whether the subject's eyes are healthy or unhealthy. In some implementations, the machine learning model may identify a possible ocular disease in the subject's eyes (e.g., a refractive error, a tumor). A notification and/or a message may thereafter be transmitted to the mobile device 4 or the stationary device 21 to indicate the output of the machine learning model.

In another aspect, the backend server 20 may facilitate storage of imagery acquired by the mobile device 4 or the stationary device 21, e.g., so that imagery does not have to be stored in memory on the mobile device 4 or the stationary device 21. For example, the backend server 20 may be communicatively coupled to a cloud server 22, which may be used to store imagery acquired by all users of the application (e.g., users of the mobile devices 4 and/or the stationary devices 21). The cloud server 22 may be part of a commercially available cloud service, such as an Amazon Web Services cloud server. In some implementations, the backend server 20 may also be communicatively coupled to a database 23. The database 23 may be used, for example, to store user account information (e.g., a username, a user password) associated with each user of the application. The database 23 may further store, for example, an index of the imagery associated with a particular user that is stored in the cloud server 22. The database 23 may be, for example, a MongoDB database. The backend server 20 may include a helpers API 32 to facilitate communication with the cloud server 22 and/or the database 23.

FIG. 3B shows a non-limiting example of a sequence of data flow between the mobile device 4, the backend server 20, and the cloud server 22. It should be appreciated that this same data flow may also occur using a stationary device 21. As shown, the user, upon initially opening the application on the mobile device 4, may enter a username (also referred to as an “alias”) and a password to access their user account. This input may be transmitted from the mobile device 4 to the backend server 20 via the data flow 40 (e.g., using an authUser( ) function call). In response, the backend server 20 may evaluate the input information. If the username and password match a user account, e.g., stored in the database 23, the backend server 20 may transmit a response with a token to provide the user of the mobile device 4 access to the user account via the data flow 41 (e.g., using a response( ) function call). If the username and password don't match a user account, the backend server 20 may transmit a response indicating the username and/or password is incorrect and/or the user account does not exist.

The user may then use the application on the mobile device 4 to acquire imagery of a subject (see, for example, Sections 2.1-2.3). After acquiring and processing imagery for evaluation, the imagery may be transmitted from the mobile device 4 to the backend server 20 via the data flow 42 (e.g., using an uploadEyeImage( ) function call). The imagery may be transmitted together with the token such that the imagery is associated with the user account. The backend server 20, in turn, may transmit the imagery to the cloud server 22 for storage via the data flow 43 (e.g., using the uploadImagetoCloud( ) function call). The cloud server 22 may store imagery for retrieval by the mobile device 4 and/or the backend server 20, thus alleviating the need to store imagery directly on the mobile device 4 or the backend server 20. In some implementations, the cloud server 22 may store the digital images in a Joint Photographic Experts Group (JPEG) format or a Portable Network Graphics (PNG) format. Thereafter, the cloud server 22 may transmit a message to the backend server 22 to indicate the imagery was successfully received and stored via the data flow 44 (e.g., using the response( ) function call).

The backend server 20 may store metadata associated with each image in memory on the backend server 20 via the data flow 45 (e.g., using the createNewEyeImageonDatabase( ) function call). The metadata may include, but is not limited to, a cloud identifier (ID) for the image on the cloud server 22, an image identifier (ID) for the image in a database stored on the backend server 20, a user account associated with the image, a date of birth of the subject, and a date. The backend server 20 may further evaluate the imagery using a machine learning model via the data flow 46 (e.g., using the evaluateEyeImage( ) function call). In some implementations, imagery may be retrieved from the cloud server 22 based on metadata stored in the database on the backend server 20. The output of the machine learning model may indicate the health of the subject's right eye and/or left eye. Based on this output, a notification and/or a message may be transmitted from the backend server 20 to the mobile device 4 to indicate (a) the imagery transmitted in data flow 42 was successful and/or (b) the output of the machine learning model (e.g., healthy, unhealthy) via the data flow 47.

The cloud server 22 may generally store imagery for different user accounts for later retrieval by the user, e.g., via a mobile device 4 or a stationary device 21, and/or the backend server 20. In some implementations, the output of the machine learning model associated with a particular image may also be stored, e.g., in the cloud server 22 or the database 23. This, in turn, may provide labeled data (e.g., a subject's eyes and an evaluation of its health) for use in subsequent retraining of the machine learning model.

As described above, the mobile device 4, which supports the application, is communicatively coupled to the backend server 20 to facilitate transmission of imagery acquired by the mobile device 4, and/or to retrieve notifications and/or messages from the backend server 20, e.g., notification that imagery transferred successfully or failed, a message regarding a preliminary diagnosis of the subject (e.g., healthy, unhealthy). Generally, the application may be adapted for operation on different mobile devices 4 and/or different operating systems on the mobile devices 4. For example, the application may run on various operating systems including, but not limited to, Google Android, Apple IOS, Google Chrome OS, Apple MacOS, Microsoft Windows, and Linux. In some implementations, the application may be downloaded by a user through an app store (e.g., the Apple App Store, the Google Play Store). Upon installing the application, the user of the mobile device 4, when executing the application, may gain access to the backend server 20. The application may further include web applications and cloud-based smartphone applications (e.g., the application isn't installed directly onto the mobile device 4, but is rather accessible through a web browser on the mobile device 4).

The one or more processors in the mobile device 4 and/or the backend server 20 may each (independently) be any suitable processing device configured to run and/or execute a set of instructions or code associated with its corresponding mobile device 4 and/or backend server 20. For example, the processor(s) may execute the application, as described in further detail below. Each processor may be, for example, a general-purpose processor, a Field Programmable Gate Array (FPGA), an Application Specific Integrated Circuit (ASIC), a Digital Signal Processor (DSP), and/or the like.

The memory of the mobile device 4, the backend server 20, the cloud server 22, and/or the database 23 may encompass, for example, a random-access memory (RAM), a memory buffer, a hard drive, a database, an erasable programmable read-only memory (EPROM), an electrically erasable read-only memory (EEPROM), a read-only memory (ROM), Flash memory, and/or so forth. The memory of the mobile device 4, the backend server 20, the cloud server 22, and/or the database 23 may store instructions that cause the one or more processors of the mobile device 4 and the backend server 20, respectively, to execute processes and/or functions associated with the application. The memory of the mobile device 4, the backend server 20, the cloud server 22, and/or the database 23 may respectively store any suitable content for use with, or generated by, the system 90 including, but not limited to, an application, and imagery acquired by the mobile device 4.

### 2. EXAMPLE PROCESSES TO ACQUIRE IMAGERY

Following below is a description of an image acquisition process to acquire imagery of a subject's eyes for evaluation of ocular diseases. The process may generally include one or more pre-capture processes to prepare the application and/or the subject to acquire imagery, one or more capture processes to acquire the imagery, and/or one or more post-capture processes to prepare the imagery for evaluation (e.g., by the backend server 20 using the machine learning model).

### 2.1 Examples of Pre-Capture Processes

The acquisition of imagery of a subject may begin with a pre-capture process. In some implementations, the pre-capture process may be an active process that involves, for example, searching for one or more landmarks on the face of a subject to facilitate acquisition of one or more images of one or more eyes of the subject.

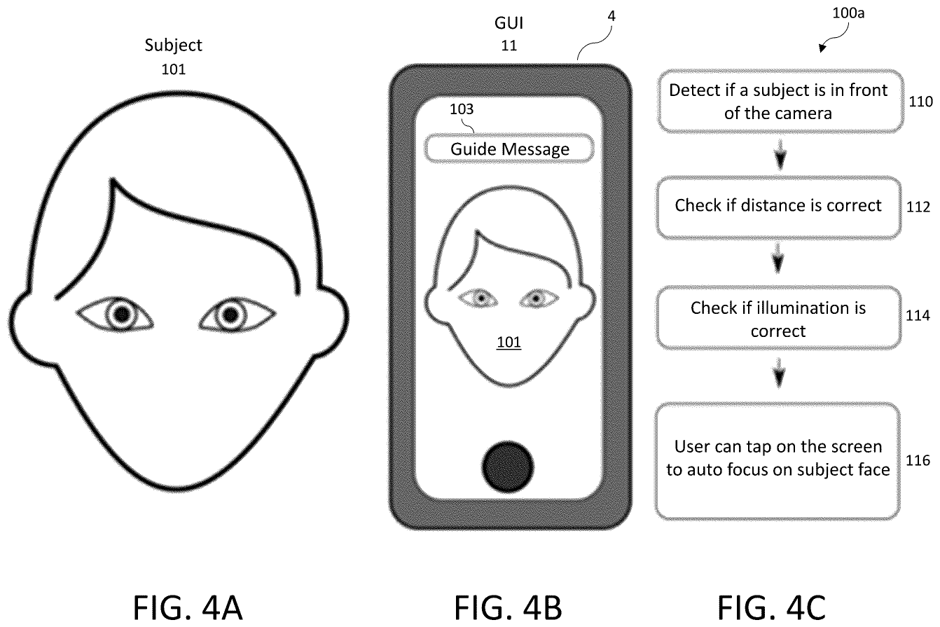

FIGS. 4A-4C show one non-limiting example of a pre-capture process. FIG. 4A shows an example subject 101. FIG. 4B shows the mobile device 4 with a graphical user interface 11 for the pre-capture process. As shown, the mobile device 4 may use the camera 1 to acquire imagery of the subject 101, which is then displayed on the graphical user interface 11. In some implementations, the camera 1 may provide a live video recording of the subject 101 to preview the imagery of the subject 101 acquired by the camera 1 so that the user may position and/or orient the mobile device 4 with respect to the subject 101 before acquiring imagery of the subject 101.

This may be facilitated, in part, by the graphical user interface 11 providing a guide feature to the user of the application (e.g., MDEyeCare). The guide feature of the application may use, for example, facial landmarks to track the position and/or orientation of the face of the subject 101 and provide one or more messages 103 to the user as to whether the subject 101 is in appropriate alignment with the mobile device 4 for image acquisition. In this manner, the guide feature may facilitate more accurate and reliable image acquisition of the subject 101.

FIG. 4C shows an example method 100a where multiple pre-capture processes are executed, in part, using the guide feature. As shown, the method may begin at step 110 with the guide feature detecting whether the subject 101 is visible by the camera 101. In some implementations, the guide feature may automatically detect a face and/or eyes in the imagery acquired by the camera 1. This may be accomplished using, for example, a Haar cascade algorithm in the application configured to detect faces and/or facial features of a person. The guide feature may display, for example, a box around the subject's face and/or each of the subject's eyes on the GUI 11 to indicate the subject's face is detected by the application. If the subject 101 is not visible in the imagery acquired by the camera 1 (e.g., the subject 101 is not in front of the camera 1), a message 103 may be displayed on the display 3 (e.g., via the graphical user interface 11) that the subject 101 is not present or not detected.

Upon detecting the face of the subject 101, the guide feature may then evaluate whether the subject 101 is at an appropriate distance from the camera 1 for image acquisition at step 112. This may be accomplished, for example, by using a depth sensor (e.g., a light detection and ranging (LiDAR) sensor) on the mobile device 4 to measure a distance between the mobile device 4 and the subject 101. Alternatively, or additionally, the distance may be estimated directly from the imagery of the subject 101.

In some implementations, it may be desirable for the distance between the subject 101 and the camera 1 to range from about 60 centimeters (cm) to about 90 cm. In some implementations, the distance may range from about 70 cm to about 80 cm. It should be appreciated that the desired distance may depend, in part, on the properties of the camera 1 (e.g., the focal length) used to acquire imagery. For example, a camera with a longer focal length may require the subject 101 to be further away from the camera. Conversely, a camera with shorter focal length may require the subject 101 to be closer to the camera. Additionally, the distance range may be shorter for a camera with a shorter depth of field. The distance range may be longer for a camera with a longer depth of field.

The guide feature may provide a message 103 if the subject 101 is too close to the mobile device 4 or too far away from the device 4. This may be accomplished by the guide feature defining a lower limit and an upper limit to the distance between the subject 101 and the device 4 and comparing the detected distance to the lower and upper limits. For example, if it is desirable for the distance to range from about 70 cm to about 80 cm, the lower limit may equal 70 cm and the upper limit may equal 80 cm.

At step 114, the guide feature may then evaluate if the illumination of the subject 101 is appropriate for image acquisition. Generally, it is preferable for the ambient lighting to be sufficiently dark so that the subject's pupils are dilated before acquiring imagery. However, it is also desirable for the ambient lighting to be sufficiently bright so that the application is able to accurately track the face of the subject 101. Accordingly, in some implementations, the illumination may be evaluated based on the luminosity of the acquired imagery. For example, the average pixel luminosity in an image may be calculated according to the following formula,

$$value = 0.299 \cdot R + 0.587 \cdot G + 0.114 \cdot B \qquad ( 1 )$$

where R, G, and B represent values of red, green, and blue, respectively for each pixel. The value may be determined for each pixel in the image. It should be appreciated that the coefficients for the R, G, and B values in Eq. (1) are non-limiting examples and that other coefficients may be used. Generally, the coefficients may range from 0 to 1 with the sum of the coefficients for the R, G, and B values being equal to 1. The values of the pixels may then be summed together and divided by the total number of pixels in the image to obtain an average pixel luminosity.

The average pixel luminosity may then be compared against preset thresholds to evaluate whether an image is too dark or too bright. For example, if R, G, and B are 8-bit parameters that have values ranging from 0 to 255, the average pixel luminosity may also range from 0 (black) to 255 (white). A lower threshold may be set to 20 and an upper threshold may be set to 70. In other words, the image may be considered to have a desired luminosity if the average pixel luminosity is from 20 to 70. It should be appreciated that the foregoing values of the lower and upper thresholds to evaluate the average pixel luminosity is a non-limiting example. More generally, the values of the lower and upper thresholds may range from 0 to 255 provided the upper threshold is greater than the lower threshold.

If the detected average pixel luminosity falls within the desired range, the lighting conditions are sufficient for the subject's pupils to dilate. In some implementations, the subject's pupils may sufficiently dilate within a few seconds after adequate lighting conditions are established.

If the detected average pixel luminosity falls outside this range, the guide feature may display a message 103 to indicate to the user that the luminosity is too dark or too bright. Thereafter, the user and/or the subject 101 may change location or adjust the lighting within the environment until the average pixel luminosity is within the desired range. In some implementations, the lower and upper thresholds may be adjusted, for example, to account for variations in skin tone, which may affect whether an image is determined to be too dark or too bright. In some implementations, the user may be provided the option to disable evaluation of the illumination (e.g., via the settings button 8).

At step 116, the application may also provide a way for the user to adjust the focus of the camera 1 (sometimes referred to herein as an “auto focus”). For example, the graphical user interface 11 may allow the user to select a portion of the imagery shown on the display 13 (e.g., by tapping the portion with their fingers) to change the focal length of the camera 1 so that it puts into focus the selected portion of the image. In another example, the application may be configured to automatically adjust the focus onto the face of the subject 101 upon detecting the subject 101 at step 110. For example, the application may periodically assess the sharpness of the subject's face and adjust the focus to increase or, in some instances, maximize the sharpness.

It should be appreciated that steps 112, 114, and 116 may be performed in any order and/or simultaneously.

### 2.2 Examples of Capture Processes

The user may begin acquiring imagery of the subject 101 using, for example, the flash 2 of the mobile device 4 to illuminate the subject's eyes in order to capture their pupillary and corneal reflexes. It should be appreciated that, in some implementations, an external flash device providing, for example, a higher intensity light source may be used with the mobile device 4 and/or the stationary device 21 to illuminate the subject's eyes (see, for example, the external flash devices 300a-300d in Section 4).

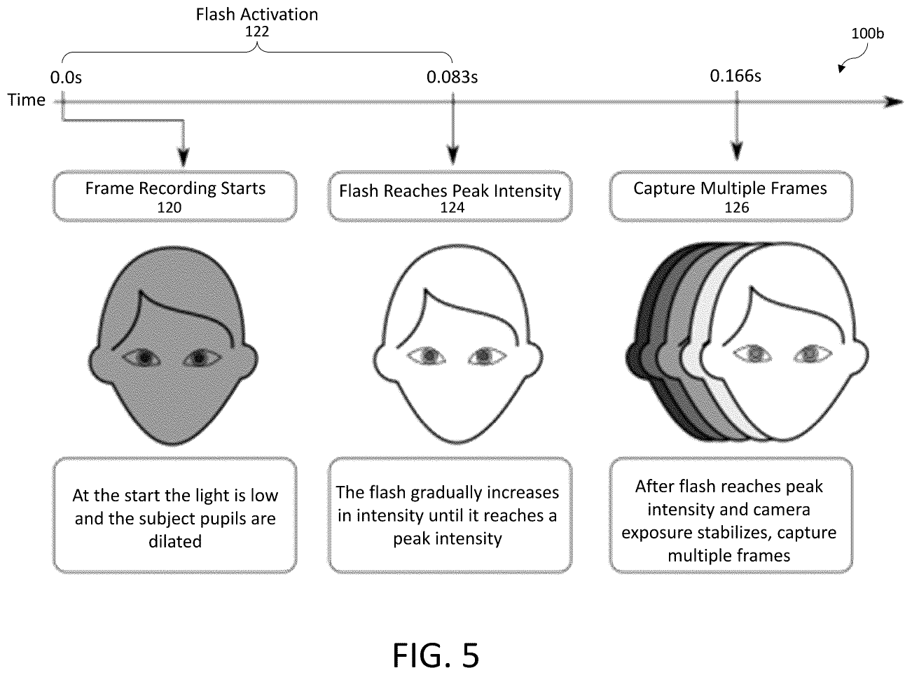

FIG. 5 shows one non-limiting example of a method 100b representing a capture process to acquire imagery of the subject 101. Upon executing the method 100b, a set of images of the subject 101 are acquired and stored in memory on the mobile device 4 for further processing.

At step 120, the capture process is initiated, for example, by the user selecting the button 7 in the graphical user interface 11. The guide feature and auto focus feature of the camera 1 may further be disabled. Additionally, the application may adjust the focus of the camera 1 during the capture process. Upon starting the capture process, the camera 1 may begin recording a video at a predetermined frame rate and a predetermined resolution. The recorded images may correspond to frames of the video. The images may further be temporarily stored in memory on the mobile device 4.

It is generally preferable for the images to be captured at a relatively higher frame rate and a relatively higher image resolution. A higher frame rate may provide corrections to the white balance and/or other corrections to the images more quickly and/or using fewer images. Additionally, a higher frame rate may reduce blurriness in the images, e.g., due to there being less motion of the subject 101 between each consecutive image. A higher frame rate may also facilitate acquisition of more images before the pupils of the subject 101 contract in response to the flash 2 of the mobile device 4. A higher image resolution may retain more detail of the subject's eyes, thus allowing for more accurate evaluation of any ocular diseases in the eyes.

However, these parameters may compete against one another. Typically, a relatively higher frame rate often requires acquiring imagery at a relatively lower resolution and vice-versa. Thus, in some implementations, the application may be configured to preferably acquire imagery at the highest image resolution possible using camera 1 and use the highest frame rate supporting that image resolution. For example, if the mobile device 4 supports recording video at 60 frames per second (fps) at an ultra-high definition (UHD) resolution (e.g., an image with 3,840 pixels by 2,160 pixels) and 120 fps at a full HD resolution (e.g., an image with 1,920 pixels by 1,080 pixels), the application may select recording video at 60 fps at a resolution of 4 k due to the higher image resolution. It should be appreciated that continued advances in camera technology may allow mobile devices to acquire imagery at higher frame rates and higher image resolutions. Accordingly, the selection of a higher frame rate at the expense of a higher image resolution or a higher image resolution at the expense of a higher frame rate is not a strict limitation.

More generally, the frame rate may range from about 30 fps to about 240 fps, including all values and sub-ranges in between. For example, the frame rate may be 30 fps, 60 fps, 120 fps, or 240 fps. The image resolution may generally be any high-definition resolution including, but not limited to, full HD (1,920 pixels by 1,080 pixels), quad HD (2,560 pixels by 1,440 pixels), and ultra HD (3,840 pixels by 2,160 pixels). It should be appreciated that the image resolution may vary depending on the size of the display 3 of the mobile device 4.

The flash 2 may turn on at step 120 or immediately thereafter. At step 122, the intensity of the flash 2 may increase gradually. A gradual increase in the intensity of the flash 2 may allow some mobile devices 4 to adjust its white balance to compensate the flash 2 in less time and/or using fewer frames compared to increasing the flash 2 to its peak intensity in a single frame. Herein, this process of increasing the intensity of the flash 2 is sometimes referred to as a “torch lit process.”

In one example, the intensity of the flash 2 may increase in increments of 20% of the peak intensity frame-to-frame. In other words, the intensity of the flash 2 may increase from 0% peak intensity, then to 20% peak intensity, then to 40% peak intensity, then to 60% peak intensity, then to 80% peak intensity, and, lastly, to 100% peak intensity across 5 successive images. If the framerate is 60 fps, the flash 2 increases from being off to its peak intensity in about 83 milliseconds (0.083 seconds).

It should be appreciated that the above example is non-limiting and that other increments may be used. The increment may generally depend on, for example, the rate at which white balance is adjusted by the mobile device 4, the frame rate, and the total time to reach peak intensity. Generally, if the total time is too long (e.g., greater than 1 second), the subject's pupils may contract before imagery is acquired by the mobile device 4.

Accordingly, the increment, in some implementations, may range from about 5% of the peak intensity of the flash 2 to 50% of the peak intensity of the flash 2, including all values and sub-ranges in between. The increment may be defined based on the desired period of time for the flash 2 to reach its peak intensity. For example, the increment may be defined such that the flash 2 reaches peak intensity from about 16 milliseconds (ms) to about 200 ms, including all values and sub-ranges in between. Preferably, the flash 2 may reach peak intensity from about 16 ms to about 100 ms, including all values and sub-ranges in between. The increment may be defined based on the desired number of frames for the flash 2 to reach its peak intensity. For example, the increment may be defined such that the flash 2 reaches peak intensity from 2 successive images to 10 successive images, including all values and sub-ranges in between. Preferably, the flash 2 may reach peak intensity from 2 successive images to 5 successive images, including all values and sub-ranges in between.

In some implementations, the rate at which the intensity of the flash 2 increases to its peak density may be non-linear. In other words, the increment in the intensity of the flash 2 may vary from frame to frame. In some implementations, the increment may increase in value over time until the peak intensity is reached. For example, the increment may follow an exponential function. In some implementations, the increment may decrease in value over time until the peak intensity is reached. For example, the increment may follow a natural log function.

Once the flash 2 reaches its peak intensity, the capture process may undergo a waiting period to allow the exposure of the camera 1 to stabilize at step 124. The images acquired by the mobile device 4 up until the end of the waiting period may be discarded. Referring to the example shown in FIG. 5, the first ten frames (i.e., the frames up to and equal the time of 166 ms assuming a 60 fps framerate) may be discarded.

In one non-limiting example, the waiting period may equal five successive images acquired at a frame rate of 60 fps, or a time period of about 83 ms. Generally, the waiting period may range from 0 ms to 200 ms, including all values and sub-ranges in between. Preferably, the waiting period may range from 0 ms to 100 ms, including all values and sub-ranges in between. Alternatively, the waiting period may range from 1 successive image to 10 successive images, including all values and sub-ranges in between. Preferably, the waiting period may range from 1 successive image to 5 successive images, including all values and sub-ranges in between.

After the waiting period, the capture process may proceed to store images acquired thereafter for possible evaluation of ocular disease at step 126. In some implementations the application may designate the stored images as “potential images” to distinguish the images from the preceding images obtained when increasing the intensity of the flash 2 and/or during the waiting period, which may be discarded after the capture process. This may be accomplished, for example, by adding metadata to each image to include a label indicating the image is a “potential image.” The images may thereafter be stored, for example, in the memory of the mobile device 4.

The number of images acquired may generally vary depending on the framerate and/or the time period to acquire the images. In particular, the time period to acquire these images should not be exceedingly long since the longer the flash 2 is on, the more the subject's pupils contract. Said another way, it is preferable for the acquisition time to be relatively short to reduce the amount of time the flash 2 is active and illuminating the subject's eyes. In one non-limiting example, ten frames may be acquired for further processing and the flash 2 is turned off thereafter. If the images are captured at a framerate of 60 fps, the time period to acquire the images is about 166 ms. Thus, in the example of FIG. 5, the total period of time for the capture process may be equal to about 330 ms (i.e., 20 frames total captured at 60 fps framerate).

More generally, the number of images acquired for potential evaluation may range from 1 image to 20 images, including all values and sub-ranges in between. In some implementations, the time period to acquire images for potential evaluation may range from about 10 ms to about 200 ms, including all values and sub-ranges in between.

In some implementations, the application may be configured to emit an audible cue at step 120 or shortly after step 120 (e.g., while the flash 2 is increasing in intensity). The audible cue may be used to attract the attention of the subject 101 to the camera 1, particularly if the subject 101 is a child or an infant. Said another way, the audible cue may be used to get the subject 101 to look at the camera 1 so that imagery of the subject's eyes may be acquired. The audible cue may be timed so that the flash 2 and camera 1 begin the process of image acquisition in tandem with the audible cue, or shortly thereafter at an appropriate time. The audible cue may continue during the capture process in some cases or, alternatively, only at the beginning of the capture process to attract the attention of the subject.

In one non-limiting example, the audible cue may be a barking dog. This example is particularly useful since it is often instinctive for a child to be attracted to the sound of a barking dog, and accordingly turn their gaze and attention to the direction where the barking sound is coming from (e.g., the speaker of the mobile device 4 used to acquire imagery). It should be appreciated that other forms of audible cues to attract the attention and gaze of the subject 101 may be employed including, but not limited to, human voice cues, other animal noises, musical tones, and portions or, in some instances, full versions of well-known songs (e.g., nursery rhymes).

### 2.3 Examples of Post Capture Processes

Once a set of images is acquired for potential evaluation, the application may execute one or more post capture processes to facilitate the selection of one (or more) images from the set of images for evaluation.

One example post capture process may discard acquired images that are either too dark or too bright. The brightness of the acquired images may vary, for example, due to sudden changes in environmental lighting during the capture process. This may be accomplished, for example, by evaluating the average pixel luminosity of the acquired images using Eq. (1). This post capture process, however, may be distinguished from the pre-capture process used to assess the illumination of the subject before image acquisition in that the subject's face in the acquired images is illuminated by the flash 2. Accordingly, the lower and upper thresholds for evaluating whether an acquired image is too dark or too bright, respectively, may be different than the lower and upper thresholds described in Section 2.1.

For example, if R, G, and B in Eq. (1) are 8-bit parameters that have values ranging from 0 to 255, the lower threshold may be set to 50 and the upper threshold may be set to 200. Thus, the image may be considered to have a desired luminosity if the average pixel luminosity is from 50 to 200. If all the acquired images fall outside the foregoing range, a message may be displayed on the graphical user interface 11 that no viable images of the subject were acquired. The user may then be provided an option to repeat the capture process. It should be appreciated that the foregoing values of the lower and upper thresholds to evaluate the average pixel luminosity is a non-limiting example. More generally, the values of the lower and upper thresholds may range from 0 to 255 provided the upper threshold is greater than the lower threshold.

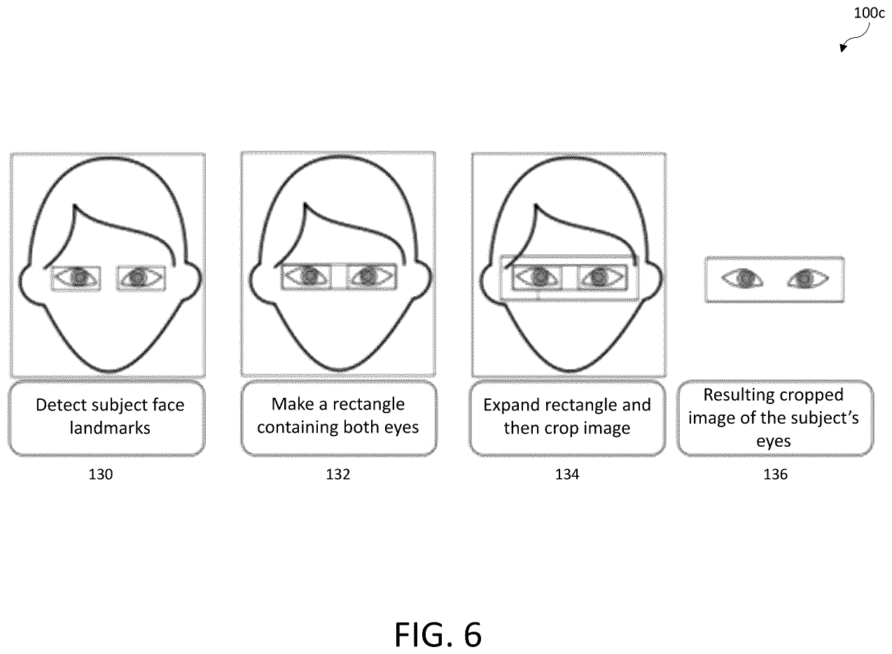

Another example post capture process may crop the acquired images, for instance, to isolate the eyes of the subject. In some implementations, this process to crop the image may follow the process described above to discard images based on their brightness. In some implementations, each of the remaining acquired images may be cropped. FIG. 6 shows an example method 100c representing this post capture process to crop an acquired image. As shown, the method 100c may begin at step 130 by detecting landmarks on the subject's face, such as their eyes. This may be accomplished, for example, using an appropriate Haar cascade algorithm configured to detect eyes in imagery. If none of the acquired images include the subject's face, a message may be displayed on the graphical user interface 11 that no viable images of the subject were acquired. The user may then be provided an option to repeat the capture process.

Once the subject's eyes are detected, a rectangle may be created to contain a subset of pixels within the image corresponding to both eyes at step 132, as shown in FIG. 6. The rectangle may, for example, be dimensioned to be tightly bound around the subject's eyes where the sides of the rectangle may intersect the outermost edges of the subject's eyes.

At step 134, the rectangle may be expanded to include a larger portion of the image around the subject's eyes. In some implementations, each side of the rectangle may be expanded by a predetermined number of rows or columns of pixels. For example, the top and bottom sides of the rectangle may extend upwards and downwards, respectively, by a predetermined number of rows of pixels (e.g., 5 rows of pixels for each of the top and bottom sides). In another example, the left and right sides of the rectangle may extend leftwards and rightwards, respectively, by a predetermined number of columns of pixels (e.g., 5 columns of pixels for each of the left and right sides).

At step 136, the image be cropped such that only the portion of the image contained within the rectangle is retained (i.e., the portion of the image located outside the image is discarded). In this example, each cropped image may show both eyes of the subject. Accordingly, a single image may be evaluated to assess the health of each of the subject's eyes.

However, it should be appreciated that, in some implementations, a pair of images may be created from each image with one image corresponding to the subject's right eye and the other image corresponding to the subject's left eye. For example, a Haar cascade algorithm may be used to isolate the right eye and the left eye in each image, which may then be cropped and stored in the pair of images. Each image may be separately evaluated to assess whether an ocular disease is present.

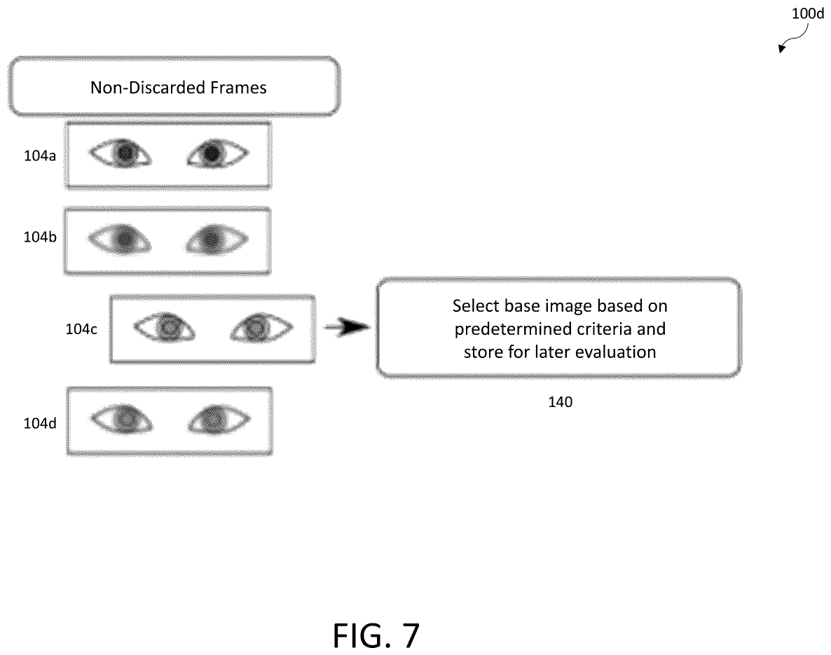

Once the acquired images are cropped, another example post capture process may select at least one image from the remaining cropped images for evaluation. In some implementations, the post capture process may be configured to select a single image from the remaining cropped images for evaluation. For example, FIG. 7 shows an example method 100d where multiple cropped images 104a, 104b, 104c, and 104d remain after the other processes described above are executed. From this remaining set of cropped images, image 104c may be selected for evaluation according to a predetermined criteria.

In one example, the predetermined criteria may include selecting the cropped image with the highest average pixel luminosity. In other words, if the process described above to discard images based on their brightness is applied, the cropped image selected according to this criteria is the cropped image with the highest average pixel luminosity that falls within the upper and lower thresholds described above.

In another example, the predetermined criteria may include selecting the cropped image with the highest sharpness. This may be accomplished, for example, by defocusing each cropped image using a Gaussian filter, and then applying a Fast Fourier Transform (FFT) to the defocused image to determine a value representing the image sharpness. It should be appreciated that, in some implementations, the criteria may include evaluating an image to assess its brightness and sharpness. Furthermore, weights may be attached to the brightness and the sharpness to give one parameter greater priority when selecting the cropped image. For example, brightness may have a weight of 0.3 and the sharpness may have a weight of 0.7 so that the sharpness is a more significant factor in the selection of an image.

In the method 100d, each of the cropped images 104a, 104b, 104c, and 104d may be analyzed according to the same criteria. The cropped image that best satisfies the criteria (e.g., the cropped image with the highest brightness and/or the highest sharpness) is selected for further evaluation. The selected cropped image may first be stored on the mobile device 4. Thereafter, the selected cropped image may be transmitted from the mobile device 4 to the backend server 20 (e.g., via the data flow 40 in FIG. 3B).

It should be appreciated that, in some implementations, one or more of the post-capture processes may be executed using the backend server 20. For example, after images are acquired for potential evaluation by the mobile device 4 (through the application), the acquired images may be transmitted to the backend server 20. The backend server 20 may thereafter execute the post-capture processes described above to select one (or more) images for evaluation.

### 3. EVALUATION OF THE SELECTED IMAGE

The systems disclosed herein may be configured to automatically evaluate images acquired of the subjects eyes using a machine learning model. As described above, the evaluation of imagery may be performed using the backend server 20. The machine learning models disclosed herein are trained to detect the presence of an ocular disease in the subject's eyes based on imagery acquired by the mobile device 4, as described in Section 2. Specifically, the health of the subject's eyes is evaluated based on the pupillary and/or corneal reflex of the subject's eyes. This information may, in turn, be used to provide a preliminary diagnosis of an ocular disease. As described in Section 1, the ocular diseases may include, but is not limited to, misalignment of the eyes (e.g., strabismus), different sizes of the eyes (e.g., anisometropy), abnormal growths in the eye (e.g., tumors), opacity of the eyes (e.g., cataract), and abnormalities in light refraction of the eyes (e.g., myopia, hyperopia, astigmatism). In some implementations, the machine learning models disclosed herein may further distinguish the health for each of the subject's eyes. For example, the machine learning model may provide an output indicating whether the subject's left eye or right eye is healthy or unhealthy (i.e., has an ocular disease).

Following below is a description of example machine learning models that may be used herein and various processes to generate and/or label training data to facilitate training of the machine learning model. Herein, several examples of a deep learning (DL) algorithm, particularly Convolutional Neural Networks (CNN) may be used for image classification.

### 3.1 A First Example Machine Learning Model

In one non-limiting example, a pre-trained semantic image segmentation model with a U-Net architecture may be used to facilitate classification of images acquired by the mobile device 4 through use of the application. Further information on this model architecture may be found in Ronneberger et al., “U-Net: Convolutional Networks for Biomedical Image Segmentation,” arXiv: 1505.04597, May 18, 2015, which is incorporated herein by reference in its entirety. In one non-limiting example, the Resnet34 model may be used (see, for example, https://models.roboflow.com/classification/resnet34). Resnet34 is a convolutional neural network with 34 layers pre-trained using the ImageNet dataset.

The Resnet34 model may be calibrated and/or otherwise fine-tuned to classify the health of the subject's eyes using a transfer learning technique. This may involve, for example, retraining the last layer of nodes, e.g., by adjusting the coefficients in each node in the output layer of the nodes in the neural network, to classify imagery of the subject's eyes as healthy or unhealthy. This may be facilitated, in part, by using training data that contains imagery of multiple subject's eyes and labels indicating whether the subject's eyes are healthy or unhealthy. In one example, the transfer learning technique may be implemented with 50 epochs of fine-tuning. The Resnet34 model may be retrained using, for example, the fast.ai Python library in conjunction with Google collab notebooks, which provides GPU acceleration (see https://www.fast.ai/). In some implementations, the model may be fine-tuned during 20 epochs. Various metrics may be used to evaluate the performance of the trained model including, but not limited to, DiceMulti and Foreground Accuracy.

The training data may include a collection of images depicting different subjects' pairs of eyes. In one non-limiting example, the images may be sourced from an MDEyeCare trial and the Internet (for single-eye and double-eye images). In one example, the labels applied to the training data may indicate whether the subject's eyes are healthy or unhealthy, as described above. The labels may further differentiate between the health of the subject's right eye or left eye. In some implementations, labels may also be applied to specify the underlying ocular disease, such as the presence of a refractive error or Leukocoria. Additionally, the images may be labeled to indicate whether the subject has a prosthesis or no eye.

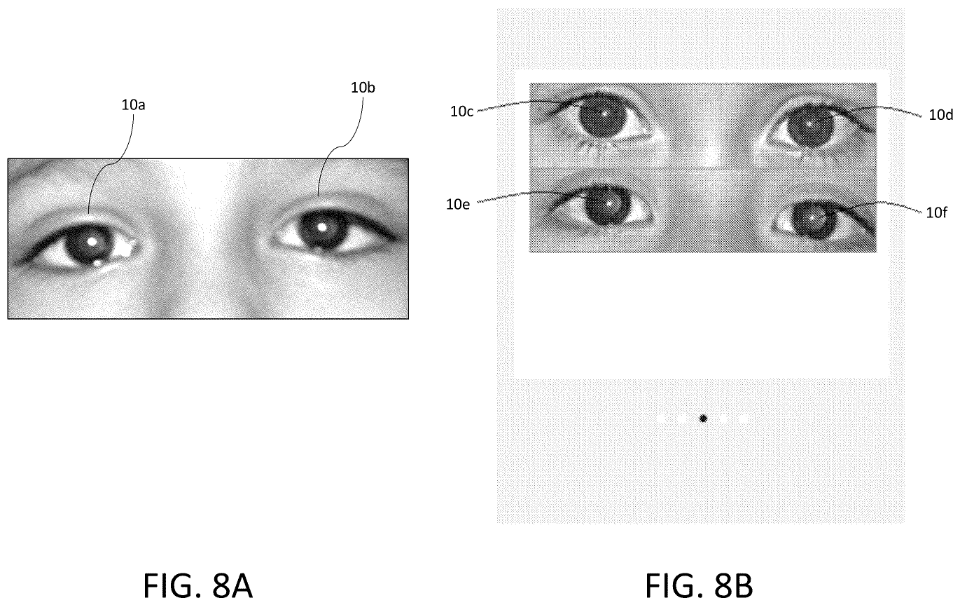

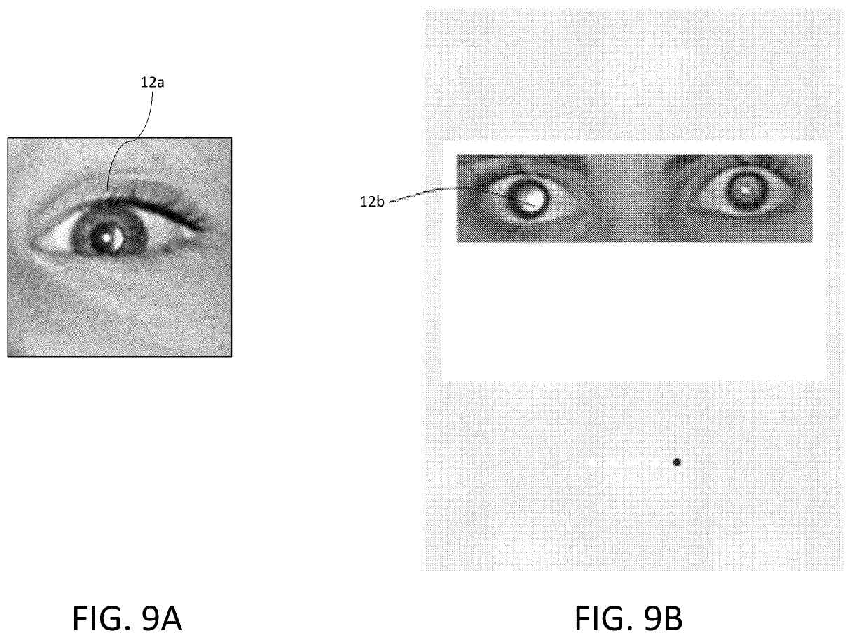

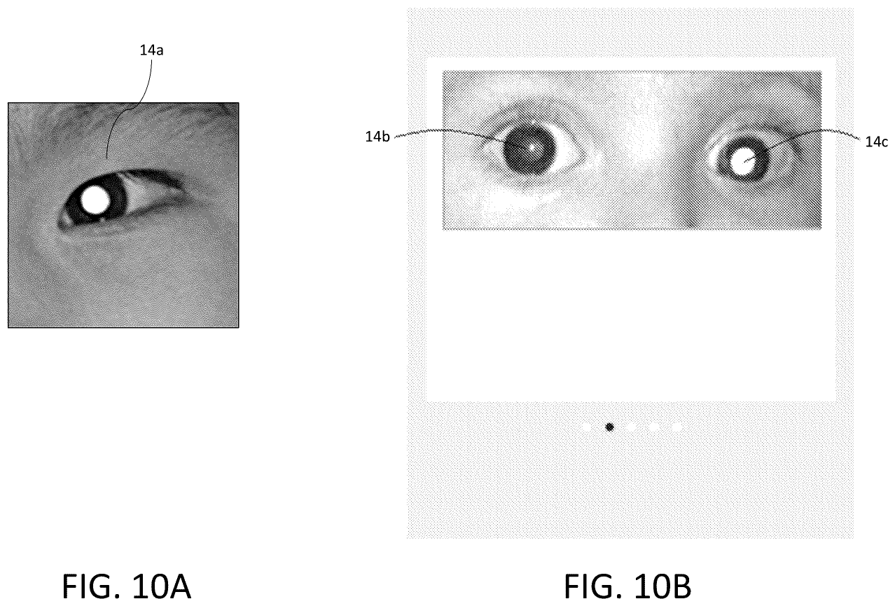

As an example, FIGS. 8A and 8B show several example images of healthy eyes (see eyes 10a, 10b, 10c, 10d, 10e, and 10f). FIGS. 9A and 9B show several example images of eyes with a refractive error (see eyes 12a and 12b). FIGS. 10A and 10B show examples of images of eyes with Leukocoria (see eye 14a) and a tumor (see eyes 14b and 14c). In one non-limiting demonstration of the machine learning model disclosed herein, the training dataset included 449 images with pairs of eyes in the Healthy category, 139 images of pairs of eyes with Refractive errors, 37 images of pairs of eyes with Leukocoria, 124 images that were sourced from the Internet depicting one or two eyes, 2 images of a subject with no eye, and 2 images of a subject with a prosthetic eye.

In regular classification models, an entire image is typically assigned to a particular class. For image segmentation models, each pixel of the image may be assigned to a particular class. The classification of the pixels in an image may be facilitated, in part, by creating a secondary image (also referred to herein as a “mask”) that indicates the class of each pixel in the original image. Typically, the creation of a mask is a manual and labor-intensive process. In the present disclosure, the process of creating a mask may be appreciably made easier and faster through use of an eye mask creator tool. The mask creator tool disclosed herein was developed in Unity. However, it should be appreciated that other development platforms may be used.

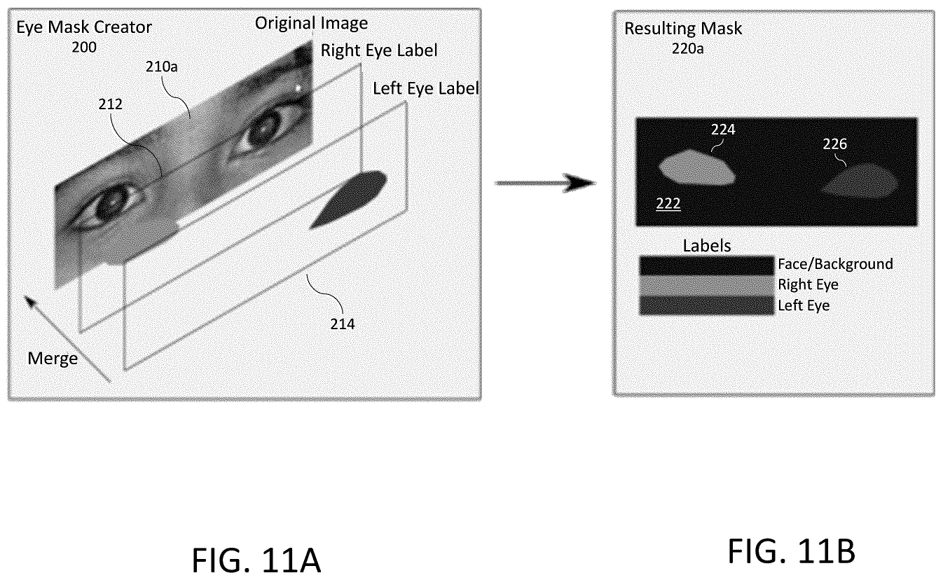

As shown in FIGS. 11A and 11B, the eye mask creator tool 200 may allow a user to draw arbitrarily shaped polygons using the original image 210a and assign labels to each polygon as desired. The polygons may then be rendered as a rasterized image with a pixel resolution equal to the original image 210a. The pixels contained within each polygon may thus be assigned a particular value (e.g., an RGB value, a grayscale value) to differentiate those pixels from different polygons with different labels.

To generate the polygons, the eye mask creator tool 200 may allow users to first create different layers corresponding to different labels to be used in the mask. For example, FIG. 11A shows that, in addition to the original image 210a, a layer 212 may be created for the subject's right eye, and a layer 214 may be created for the subject's left eye. The user may select the layer 212 and proceed to draw a polygon around the subject's right eye. Similarly, the user may select the layer 214 and proceed to draw a polygon around the subject's left eye.

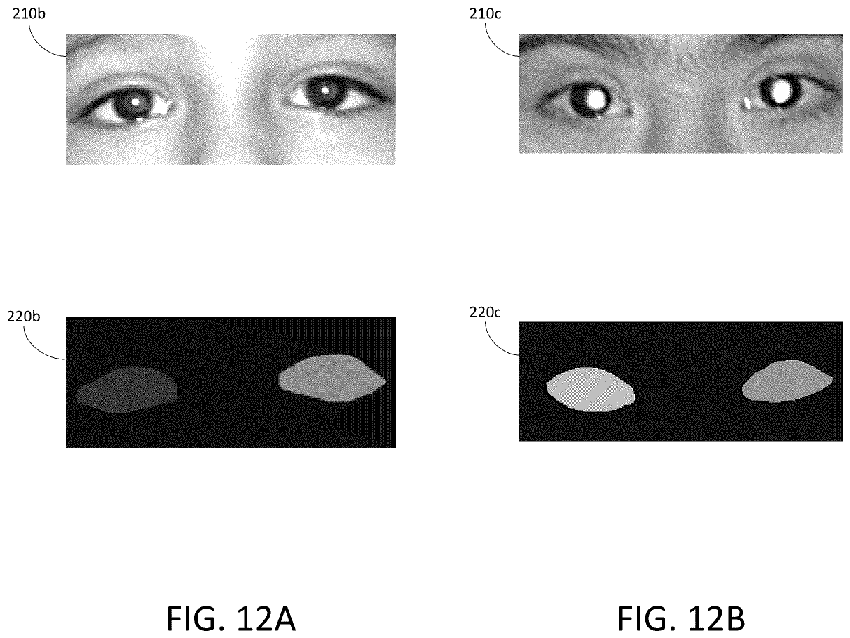

Thereafter, the layers 212 and 214 may be merged into a single composite image referred to as a mask 220a, as shown in FIG. 11B. When generating the mask 220a, the polygons contained within each layer may be rendered into pixel form. The polygons in each layer may be assigned a particular RGB value or grayscale value. The eye mask creator tool may set each layer to have a different RGB value or grayscale value to differentiate different labels in the mask 220a. In some implementations, a grayscale value may be preferable to simplify and/or reduce the size of the mask. For example, if the mask is an 8-bit grayscale image, each pixel may be assigned a single value ranging from 0 to 255. This, in turn, results in a mask having an N×M matrix of 8-bit values. In contrast, if the mask is an 8-bit colored image, each pixel may be assigned three values for R, G, and B, each of which may range from 0 to 255, thus resulting in the mask having an N×M×4 tensor of 8-bit values, which is larger in size. FIGS. 12A and 12B show additional examples of images 210b and 210c, respectively, with corresponding masks 220b and 220c represented using grayscale values.

Each polygon may be mapped onto corresponding pixels in the mask 220a that overlap and/or are contained within that polygon. That way, the label of a right eye or a left eye may have a direct correspondence to the pixels in the original image 210a. As shown in FIG. 11B, the mask 220a may, by default, include a background label 222 to cover portions of the original image 210a that are not directly labeled by the user (e.g., via the layers 212 and 214). In this case, the background label 222 may correspond to the subject's face and/or any background environment. In this example, the background label 222 is shown in blue, the right eye label 224 is shown in orange, and the left eye label 226 is shown in red.

Once the mask 220a is created, it may be stored as an image file (e.g., in PNG format). The mask 220a may then be associated with the original image 210a. For example, the mask may have a file name (e.g., “image1_mask”) that corresponds to the file name of the original image 210a (e.g., “image1”). When the original image 210a is used for training, the mask 220a may also be retrieved (e.g., from memory of the computer or server performing the training of the machine learning model) based on the file name. Thereafter, a space-separated text file may be generated from the mask 220a that contains, for example, the labels contained in the mask 220a (e.g., the labels 222, 224, and 226). In some implementations, the label assigned to each pixel in the mask 220a may also be extracted (e.g., where the label is denoted by a unique number). The text file may be used, for example, to perform various processing to the original image 210a (e.g., resizing, padding, etc.) before being passed along as training data to train the machine learning model.

Generally, the eye mask creator tool may provide users flexibility to define an arbitrary number of labels with layers for each label and/or draw an arbitrary number of polygons in each layer. The number of layers and/or polygons may vary depending, for example on the image content and/or the desired number of labels to use to disambiguate different features of the subject's eyes. In the examples shown in FIGS. 11A-12B, the labels that applied when creating a mask may include, but are not limited to, background, a healthy right eye, an unhealthy right eye, a healthy left eye, and an unhealthy left eye.

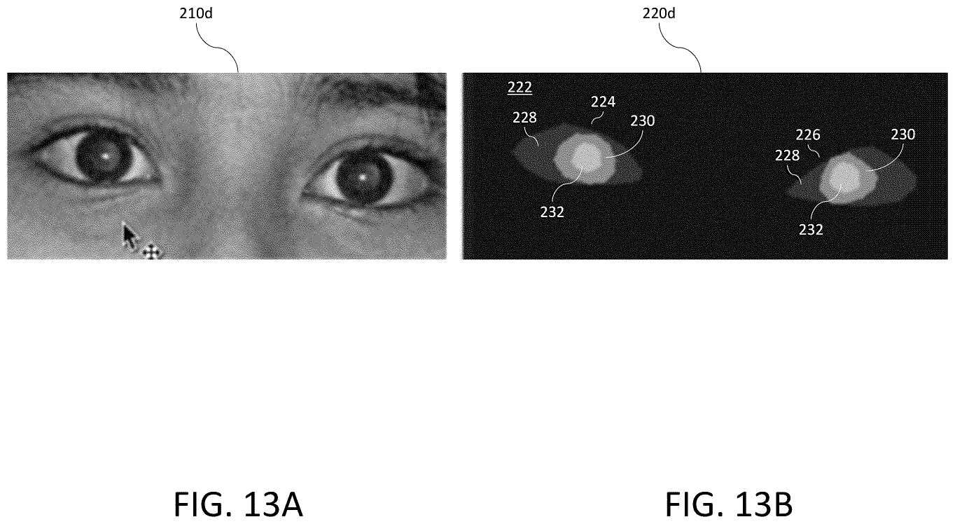

FIGS. 13A and 13B show another example where additional labels are applied to different portions of the subject's eyes. As shown, the mask 220d corresponding to the image 210d includes the background label 222, the right eye label 224, and the left eye label 226 as before. Additionally, the mask 220d may include a sclera label 228, an iris label 232 and a pupil label 230 for both the right eye and the left eye. The addition of these labels may improve the performance of the machine learning by providing greater specificity to the features in the image that are important to diagnose an ocular disease. In another example, additional labels may be applied to differentiate specific conditions of the subject's eyes, such as a label for a refractive error, Leukocoria, a tumor, a prosthetic eye, no eye, and so on.

In some implementations, the various labels in the mask may be used to obtain additional information on the subject's eyes when evaluating imagery of the eyes using the trained machine learning model. For example, the machine learning model may output the location of the subject's eyes, the sclera, the iris, the pupil. It should be appreciated, however, that this additional information may be used in evaluating the presence of any ocular disease.

It should also be appreciated that the machine learning models disclosed herein may be periodically or, in some instances, continually retrained over time, particularly as more data is acquired by users using the application and the system. For example, the imagery stored in the cloud server 22 by different users may be used to periodically retrain the machine learning model. The outputs generated by the machine learning model (e.g., healthy, unhealthy) may be used to label the imagery for training purposes. In some implementations, the application may also allow the user and/or the subject to provide feedback, for example, to confirm or deny the diagnosis provided by the machine learning model. For example, if the application indicates a subject may have an ocular disease and later discovers the diagnosis is incorrect (e.g., after visiting a specialist), the application may allow the subject to correct the diagnosis in the application for that image. In this manner, corrections to the outputs of the machine learning model may be incorporated as training data to retrain the machine learning model.

### 3.3 A Second Example Machine Learning Model

In another non-limiting example, a pre-trained image classification model with a ResNet architecture may be used to facilitate classification of images acquired by the mobile device 4 through use of the application. Further information on this model architecture may be found in He et al., “Deep Residual Learning for Image Recognition,” arXiv:1512.03385, Dec. 10, 2015, which is incorporated by reference herein in its entirety. This model may also use the Resnet34 model as a starting point, as described in Section 3.2. A transfer learning technique may also be applied to fine tune this model to classify images related to a subject's eyes to assess the presence of ocular disease. The transfer learning technique may be applied in a similar manner as described in Section 3.2. For brevity, repeated discussion of this technique is not provided below.

For this model, training data may be generated using an eye Haar Cascade classifier (e.g., in the OpenCV library) to create a set of images (also referred to as “stamps”) that show one eye from the images acquired by the mobile device 4. In executing this process, metadata for each acquired image may be removed, and the number of images available for training may be appreciably increased. Thereafter, the stamps may be resized to 128 pixels×128 pixels for training.

Each stamp may be stored in a folder named according to its label (e.g., healthy, unhealthy, healthy right eye, unhealthy right eye, healthy left eye, unhealthy left eye, refractive error, Leukocoria, tumor, no eye, prosthetic eye). In some implementations, the process of labeling the eyes with a particular condition may be accomplished using a specialist (e.g., an ophthalmologist) to evaluate whether a subject's eye has an ocular disease.

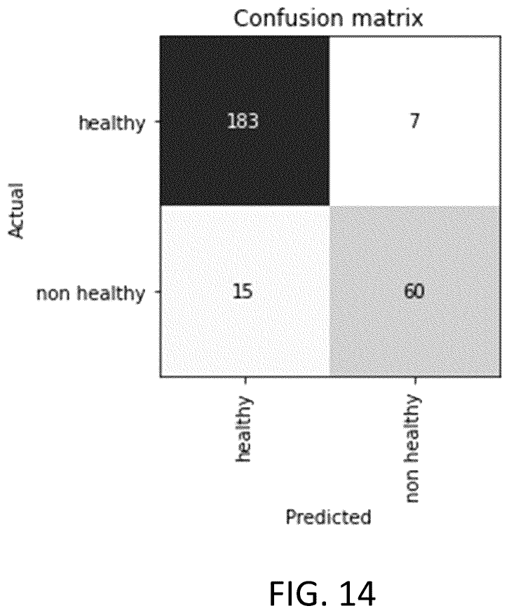

With this training data, the transfer learning technique was used to retrain the ResNet34 model with 50 epochs of fine-tuning. In some implementations, the best-performing model may be selected for deployment based on an error rate metric. With this approach, a model with a success rate of 92.8% was achievable (see, for example, the confusion matrix in FIG. 14).

### 4. EXAMPLES OF EXTERNAL FLASH DEVICES

The quality of images acquired showing the pupillary reflex and/or the corneal reflex of a subject's eyes may vary between different types of mobile devices 4 (e.g., different smart phone models) due, in part, to the variable placement of the flash 2 with respect to the camera 1. In some instances, certain models of mobile devices 4 may be unable to acquire images that adequately capture the pupillary reflex and/or the corneal reflex of a subject's eyes. Moreover, the limited intensity of the light emitted by the flash 2 of a conventional mobile device 4 may limit the amount of light reflected by the subject's eyes that is captured in the image to show the subject's pupillary and corneal reflex. This, in turn, may make it more challenging to accurately assess the health of the subject's eyes.

Accordingly, in some implementations, an external flash device may be coupled to the mobile device 4 to provide a light source that may be better placed to the camera 1 and provide higher intensity light to facilitate acquisition of higher quality images of the subject's eyes. The external flash device may, for example, be directly mounted to the mobile device 4 and used as a replacement for the flash 2. Thus, the external flash device may be used together with the camera 1 of the mobile device 4 to acquire imagery of the subject's eyes.

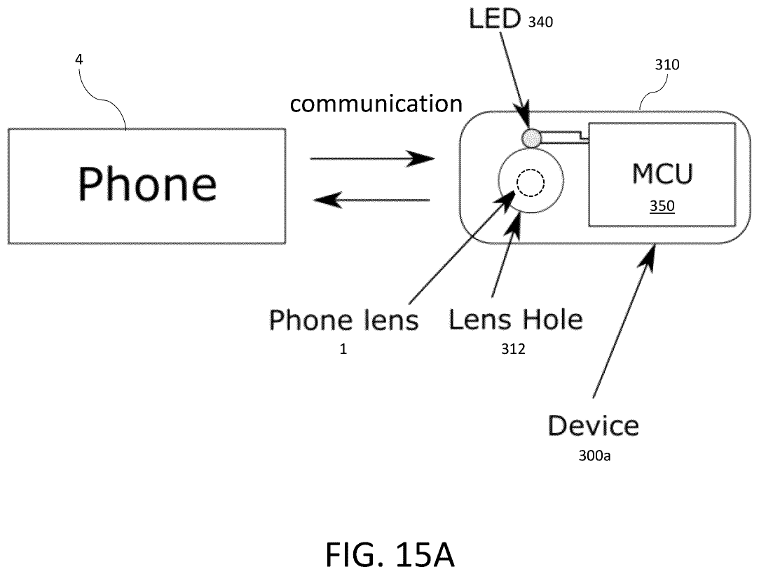

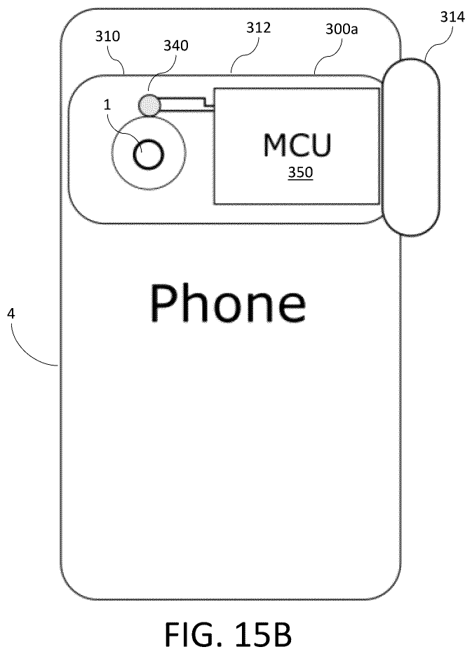

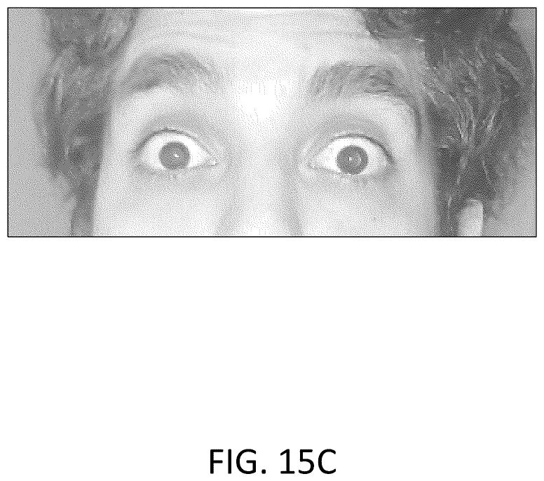

FIG. 15A shows an example external flash device 300a. As shown, the external flash device 300a may include a frame 310 that defines an aperture 312 (also referred to herein as a “lens hole 312”) through which the camera 1 of the mobile device 4 may acquire imagery. A light source 340 (e.g., an LED) may be mounted to the frame 310 in close proximity to the aperture 312. The light source 340 may function as a flash to facilitate the acquisition of images of the subject when used in combination with the mobile device 4 and the application. The device 300a may further include a microcontroller unit (MCU) 350 to manage operation of the light source 340 and to facilitate communication with the mobile device 4. For example, the MCU 350 may support one or more wireless communication protocols to communicate with the mobile device 4 including, but not limited to, Wi-Fi (e.g., Wi-Fi 802.11n) and Bluetooth. In this manner, the device 300a may be communicatively coupled to the mobile device 4 such that, during operation, the application executed on the mobile device 4 may activate or deactivate the light source 340 on demand (e.g., when acquiring imagery of the subject). As an illustrative example, the MCU 350 may be an Espressif ESP32 or an Espressif ESP8266.

In some implementations, the device 300a may further include a power supply (e.g., a rechargeable battery) to provide electrical power to the device 300a. In some implementations, the device 300a may receive electrical power from the mobile device 4. For example, the mobile device 4 may be connected to a charger port of the mobile device 4 (e.g., a charge port) using a cable. The frame 310 may support a charger port electrically coupled to the MCU 350 for connection to the cable. In another example, the device 300a may receive electrical power wirelessly, e.g., using a wireless power receiver integrated into the frame 310, which is configured to receive power from a wireless power transmitter on the mobile device 4.

The device 300a may further include various electronic components including, but not limited to, a resistor, a transistor (e.g., a MOSFET), and a switch, to facilitate operation of the device 300a. In some implementations, the device 300a may include one or more transistors for use as a switch to turn on or off the light source 340. This approach may be preferable when, for example, the light source 340 uses an electric current for operation that is appreciably greater than the electric current supported at any connection with the MCU 350. For example, the MCU 350 may transmit a low electric current signal to switch a transistor, causing a high electric current signal originating from the power supply to be transmitted directly to the light source 340.

In conventional flash devices, the operation of the device is typically facilitated by an operating system of the mobile device 4. The activation or deactivation of a flash device typically requires a trigger command originating from the operating system via a hook. For example, the conventional flash device may not turn on until it receives a trigger command from the operating system indicating an image is being taken by the camera 1. As a result, the responsiveness of conventional flash devices may vary appreciably between different operating systems, different versions of the same operating system, and/or the operating status of an operating system at any given moment in time. In some instances, conventional flash devices may experience delays in activation exceeding 1 second. Moreover, certain operating systems may restrict when a conventional flash device is activated or deactivated, such as only when recording a video or when taking an image.

Compared to conventional flash devices (e.g., the flash 2, an external flash device), the flash device 300a may be appreciably more responsive in that activation or deactivation of the light source 340 may occur faster (e.g., less than or equal to 200 ms) and/or more predictably in response to a trigger command (e.g., a response repeatedly occurs 150 ms after the command from the application is transmitted to the device 300a). This may be accomplished, for example, by the flash device 300a being configured so that it does not rely upon any hooks or triggers from the operating system of the mobile device 4 for operation. In other words, when the flash device 300a is communicatively coupled to the mobile device 4 using, for example, a Bluetooth connection, the mobile device 4 may view the device 300a as a standard Bluetooth device capable of communicating with the application. If the application generates a command to turn on the light source 340, the command may be transmitted directly from the application to the device 300a without waiting for a separate trigger command from the operating system. In this manner, the delay between the application generating a command to turn on (or off) the light source 340 and the light source 340 turning on (or off) may be appreciably reduced. In some implementations, the delay may be limited by the communication protocol used to facilitate communication between the device 300a and the mobile device 4. For example, the delay may be limited to the latency of Bluetooth communication, e.g., less than 200 ms.

The light source 340 may provide a relatively higher intensity light source compared to conventional flashes integrated into the mobile device 4. In one non-limiting example, the light source 340 may be a 1 W white LED that provides a color temperature of 6500K to 7000K and operates using a voltage of 3.2 V to 3.4 V and a current of about 400 mA.

The light source 340 may generally be disposed in close proximity to the aperture 312 so that the light source 340 emits more light that is nearly coaxial or coaxial with the camera 1. This, in turn, may increase the amount of light reflected from the subject's eyes—in particular, the subject's pupils—for collection by the camera 1, thus increasing the strength of the pupillary and/or corneal reflex captured in an image (see, for example, an example image acquired by a prototype external flash device 300a in FIG. 15C). Additionally, increasing the amount of light that is emitted nearly coaxial or coaxial to the camera 1 may allow higher quality images to be taken with the subject located closer to the camera 1 since more light may enter the subject's pupils along a direction that is parallel to the centerline of the camera 1. In some implementations, the distance separating the light source 340 and the lens of the camera 1 may be less than or equal to about 1 cm. Preferably, the distance separating the light source 340 and the lens of the camera 1 may be less than or equal to about 0.5 cm. More preferably, the distance separating the light source 340 and the lens of the camera 1 may be less than or equal to about 0.1 cm.

Although FIG. 15A shows the device 300a may include a single light source 340, it should be appreciated that this is a non-limiting example. More generally, the device 300a may include one or more light sources. For example, two or more light source 340 may be disposed around the aperture 312 to provide more light that is nearly coaxial or coaxial with the camera 1 to further increase the amount of light collected by the camera 1 from the subject's pupils. The multiple light sources may be distributed evenly around the aperture 312 (e.g., two light sources may be disposed diametrically opposite one another). In some implementations, the light source 340 may shaped as a ring that is disposed around the aperture 312.

In some implementations, the frame 310 may be configured for a particular mobile device 4 such that, when the device 300a is attached to the mobile device 4, the aperture 312 is aligned to the camera 1 of the mobile device 4. If the mobile device 4 includes multiple cameras, the aperture 312 may be aligned to only one of the cameras. For example, the camera selected may provide a focal length of about 70-80 cm. In some implementations, the other cameras of the mobile device 4 may not be used. For example, FIG. 15B shows the device 300a may be coupled to the mobile device 4 via an attachment mechanism 314. The attachment mechanism 314 may include, but is not limited to, a clamp, a clip, or a fastener to fasten the device 300a directly to the mobile device 300a. When the device 300a is attached to the mobile device 4, the portion of the frame 310 defining the aperture 312 may physically contact the portion of the mobile device 4 surrounding the camera 1. The diameter of the aperture 312 and/or the thickness of the frame 310 may be dimensioned such that the frame 310 does restrict and/or otherwise occlude the field of view of the camera 1.

In some implementations, the external flash devices disclosed herein may be configured for use with different mobile devices 4 having different cameras and/or different arrangements of cameras. For example, the frame 310 may accommodate different mobile devices 4 by providing a way to adjust the position of the aperture 312 and the light source 340 with respect to the mobile device 4. In another example, the aperture 312 may be dimensioned to accommodate cameras with different-sized lenses. For instance, the aperture 312 may be dimensioned to have a diameter of about 8 mm to accommodate cameras with lenses that have a diameter less than or equal to 8 mm. This may be accomplished in several ways.

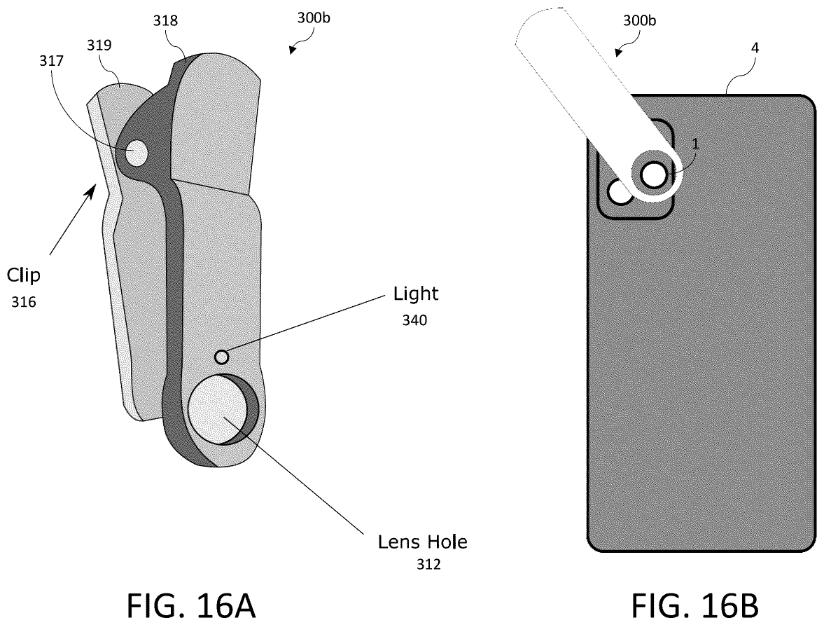

In one example, FIG. 16A shows a device 300b that includes a frame 310 with a clip 316. As shown, the clip 316 may include an arm 317 coupled to an arm 318 via a pin joint 319. The pin joint 319 may further include a spring (not shown). The spring may provide a clamping force to attach the device 300b to the mobile device 4, as shown in FIG. 16B. The end of the arm 318 may further include the aperture 312 and the light source 340. As shown in FIG. 16B, the device 300b may be attached to the mobile device 4 with the aperture 312 aligned to any one camera of the multiple cameras of the mobile device 4.

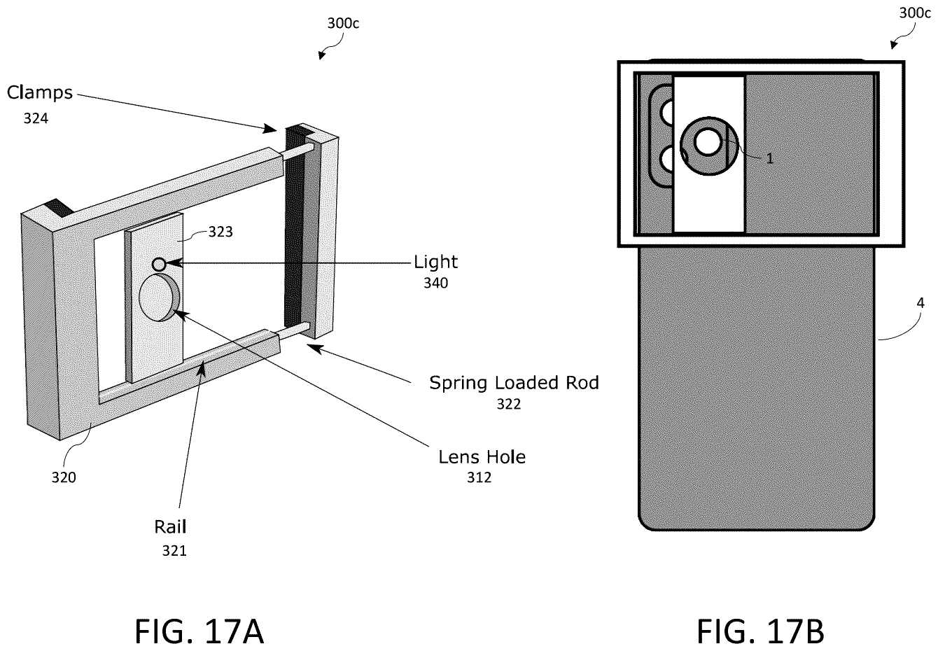

FIG. 17A shows another example device 300c that allows adjustment of the aperture 312 and the light source 340 along a single axis. As shown, the device 300c may include a clamping portion 324 coupled to a frame 320 via a spring-loaded rod 322, thus forming a spring-loaded clamping mechanism to mount the device 300c to the sides of the mobile device 4, as shown in FIG. 17B. The device 300c may further include a bridge 323 slidably coupled to the frame 320 via one or more rails 321. The bridge 323 may further define the aperture 312 and support the light source 340. Thus, the device 300c provides a way to adjust the position of the aperture 312 and the light source 340 along one axis to accommodate cameras that are placed at different positions along the axis. In some implementations, the bridge 323 may be secured in place due to the friction between the bridge 323 and the rails 321. In other words, the bridge 323 may only be slidably positioned when a sufficiently large external force (e.g., by the user) is applied to move the bridge 323.

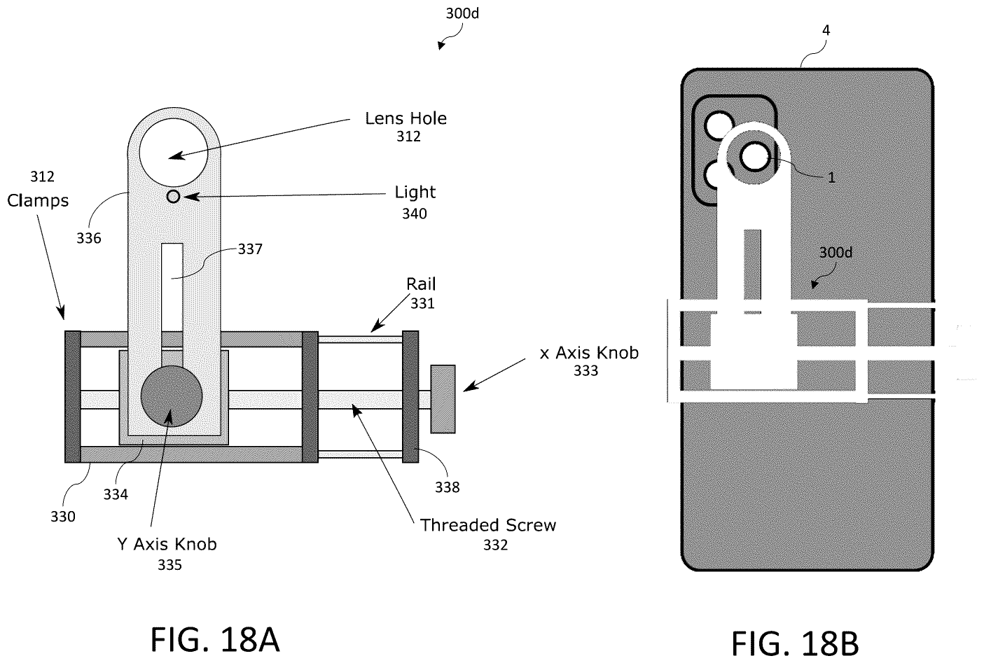

FIG. 18A shows another example device 300d that allows adjustment of the aperture 312 and the light source 340 along two axes. As shown, the device 300d may include a frame 330 and a clamping portion 338 slidably coupled to the frame 330 via one or more rails 331. The clamping portion 338 may be further coupled to the frame 330 via a spring-loaded rod. The device 300d further includes a mounting block 334 that is slidably coupled to the frame 330 via a fastener 333 that includes a knob. The device 300d further includes an arm 336 slidably coupled to a fastener 335 that includes a knob. In particular, the arm 336 includes a slot 337. The fastener 335 may pass through the slot 337 and be inserted into an opening on the mounting block 334 to securely couple the arm 336 to the mounting block 334.

The fastener 333 may thus be used to adjust the position of the arm 336 and, by extension, the aperture 312 and the light source 340 along an X axis. For example, the fastener 333 may be a threaded fastener and rotation of the knob 333 may translate the mounting block 334 along the X axis. The position of the arm 336 may further be adjustable along a Y axis by loosening the fastener 335 and slidably moving the arm 336 relative to the fastener 335 along the slot 337. The position of the arm 336 along the Y axis may be secured by tightening the fastener 335.

### 5. CONCLUSION

All parameters, dimensions, materials, and configurations described herein are meant to be example and the actual parameters, dimensions, materials, and/or configurations will depend upon the specific application or applications for which the inventive teachings is/are used. It is to be understood that the foregoing embodiments are presented primarily by way of example and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein.

In addition, any combination of two or more such features, systems, articles, materials, kits, and/or methods, if such features, systems, articles, materials, kits, and/or methods are not mutually inconsistent, is included within the inventive scope of the present disclosure. Other substitutions, modifications, changes, and omissions may be made in the design, operating conditions and arrangement of respective elements of the example implementations without departing from the scope of the present disclosure. The use of a numerical range does not preclude equivalents that fall outside the range that fulfill the same function, in the same way, to produce the same result.

The above-described embodiments can be implemented in multiple ways. For example, embodiments may be implemented using hardware, software or a combination thereof. When implemented in software, the software code can be executed on a suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in a suitable form, including a local area network or a wide area network, such as an enterprise network, an intelligent network (IN) or the Internet. Such networks may be based on a suitable technology, may operate according to a suitable protocol, and may include wireless networks, wired networks or fiber optic networks.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine. Some implementations may specifically employ one or more of a particular operating system or platform and a particular programming language and/or scripting tool to facilitate execution.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All publications, patent applications, patents, and other references mentioned herein are incorporated by reference in their entirety.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A non-transitory computer-readable medium having a plurality of instructions encoded thereon that, when executed by at least one processor, cause the at least one processor to perform a method for facilitating a preliminary diagnosis of an ocular disease in a subject by acquiring successive images of the subject's eyes, the method comprising:
A) controlling an imaging device to acquire a first number of the successive images of the subject's eyes, wherein the subject's eyes initially are sufficiently dilated;
B) during A), controlling a light source so as to increase an amount of illumination generated by the light source and directed at the subject's eyes gradually from zero to a non-zero amount that causes sufficient reflection of the illumination generated by the light source from the subject's eyes to the imaging device to effectively render the subject's eyes in at least some of the successive images;
C) after the illumination generated by the light source reaches the non-zero amount so as to cause the sufficient reflection, controlling the imaging device to acquire a second number of the successive images, after acquiring the first number of the successive images and while the illumination generated by the light source has at least the non-zero amount to cause the sufficient reflection;
D) controlling at least the imaging device to acquire at least the first number of the successive images in A) and the second number of the successive images in C) for an amount of time that is sufficiently limited to effectively mitigate significant pupil contraction of the subject's eyes during C); and
E) controlling a memory to store the second number of the successive images.

2. The computer readable medium of claim 1, wherein C) comprises, after the illumination generated by the light source reaches the non-zero amount to cause the sufficient reflection:
C1) acquiring the second number of the successive images via the imaging device, after the first number of the successive images, while the illumination generated by the light source has at least the non-zero amount to cause the sufficient reflection;
C2) acquiring a third number of the successive images via the imaging device, after the first number of successive images and before the second number of the successive images, while the illumination generated by the light source has at least the non-zero amount to cause the sufficient reflection; and
C3) discarding the third number of the successive images to allow an exposure of the imaging device to stabilize,
wherein the amount of time that the imaging device is operated to acquire at least the first number of the successive images, the second number of the successive images, and the third number of successive images is sufficiently limited to effectively mitigate significant pupil contraction of the subject's eyes during C1).

3. The computer readable medium of claim 2, wherein:
in B), the first number of successive images is five;
in C1), the second number of successive images is ten; and
in C2), the third number of successive images is five.

4. The computer readable medium of claim 1, wherein the amount of time in D) that the imaging device is controlled in to acquire the successive images is less than or equal to approximately 330 milliseconds.

5. The computer readable medium of claim 1, wherein B) comprises:
increasing the amount of the illumination by a same portion from one image to a next image of the first number of the successive images, wherein the same portion is equal to the non-zero amount divided by the first number.

6. The computer readable medium of claim 5, wherein in B):
the first number of successive images is five;
the non-zero amount equals a maximum illumination generated by the light source; and
the same portion is 20% of the maximum illumination.

7. The computer readable medium of claim 1, wherein the successive images of the subject's eyes are successive frames of a video and wherein:
A) comprises controlling the imaging device to acquire the first number of the successive frames of the video of the subject's eyes;
C) comprises controlling the imaging device to acquire the second number of the successive frames of the video after acquiring the first number of the successive frames and while the illumination generated by the light source has at least the non-zero amount to cause the sufficient reflection; and
D) comprises controlling at least the imaging device to acquire at least the first number of the successive frames in A) and the second number of the successive frames in C) for the amount of time that is sufficiently limited to effectively mitigate significant pupil contraction of the subject's eyes during C).

8. The computer readable medium of claim 1, wherein the imaging device includes a display and wherein, prior to A), the method comprises:
A-1) controlling the imaging device to provide a guide on the display, the guide including at least one notification of at least one of:
a presence of the subject in front of the imaging device;
a distance of the subject from the imaging device; or
an overall image illumination.

9. The computer readable medium of claim 8, wherein the method further comprises:
controlling the imaging device so as to disable the guide during at least A), B), and C).

10. The computer readable medium of claim 1 wherein, prior to A) and/or during A), the method comprises:
A-2) controlling at least one audio device to provide at least one audible alert to attract the attention of the subject toward the imaging device.

11. The computer readable medium of claim 10, wherein in A-2), the at least one audible alert includes at least one of:
at least one animal noise;
at least one human voice cue; or
at least one musical tone.

12. The computer readable medium of claim 11, wherein in A-2), the at least one audible alert includes at least one of:
a barking dog; or
at least a portion of a nursery rhyme.

13. The computer readable medium of claim 1, wherein the imaging device includes an auto focus function and a display and wherein the method further comprises:
prior to A), controlling the imaging device to enable the auto focus function to focus an image of the subject at a touch point on the display; and
controlling the imaging device so as to lock the auto focus function during at least A), B) and C).

14. The computer readable medium of claim 1, wherein the method further comprises:
controlling the light source so as to reduce the illumination generated by the light source to zero after C).

15. The computer readable medium of claim 1, wherein the method further comprises:
processing the second number of successive images to analyze a light content in each image of the second number of successive images; and
discarding any image of the second number of successive images in which the analyzed light content is excessive or insufficient.

16. The computer readable medium of claim 1, wherein the method further comprises:
for at least some successive images of the second number of successive images, processing each successive image of the at least some successive images to crop a rectangle containing the subject's eyes to generate a plurality of cropped images.

17. The computer readable medium of claim 16, wherein the method further comprises:
processing the plurality of cropped images to select one cropped image of the plurality of cropped images based at least in part on at least one of:
a luminosity of each cropped image of the plurality of cropped images; or
a sharpness of each cropped image of the plurality of cropped images; and
controlling the memory to store the one selected cropped image.

18. An apparatus, comprising:
the non-transitory computer readable medium of claim 1;
the at least one processor; and
the imaging device.

19. The apparatus of claim 18, further comprising the light source.

20. A system, comprising:
the apparatus of claim 18; and
the light source mechanically coupled to the apparatus such that the illumination generated by the light source is positioned proximate to the imaging device of the apparatus.

21. The system of claim 20, wherein the light source comprises:
a microcontroller communicatively coupled to the at least one processor of the apparatus;
at least one power supply; and
a frame to facilitate mechanical coupling of the light source to the apparatus.

22. The system of claim 21, wherein the frame of the light source includes a lens hole substantially aligned with the imaging device of the apparatus when the light source is mechanically coupled to the apparatus.

23. The system of claim 20, wherein the light source further comprises:
at least one wireless communication device to facilitate communicative coupling between the microcontroller and the at least one processor of the apparatus.

24. The system of claim 20, wherein the light source further comprises:
at least one light emitting diode (LED) to provide the illumination.

25. The system of claim 20, wherein the apparatus is a smartphone.

26. A method, comprising:
transmitting the plurality of instructions to the non-transitory computer readable medium of the apparatus of claim 18.

27. The method of claim 26, further comprising:
before transmitting the plurality of instructions, training a convolutional neural network (CNN) model based on a plurality of training images including a first plurality of images of healthy eyes and a second plurality of images of unhealthy eyes, wherein each training image of the plurality of images includes a mask that labels each pixel in each training image with one label of a plurality of labels; and
after E), processing at least one image from the second number of successive images using the trained CNN to determine the preliminary diagnosis of the ocular disease.

28. A method for facilitating a preliminary diagnosis of an ocular disease in a subject by acquiring successive images of the subject's eyes, the method comprising:
transmitting computer-readable instructions to an apparatus including at least one processor, wherein the computer-readable instructions, when executed by the at least one processor, cause the at least one processor to:
A) control an imaging device to acquire a first number of the successive images of the subject's eyes, wherein the subject's eyes initially are sufficiently dilated;
B) during A), control a light source so as to increase an amount of illumination generated by the light source and directed at the subject's eyes gradually from zero to a non-zero amount that causes sufficient reflection of the illumination generated by the light source from the subject's eyes to the imaging device to effectively render the subject's eyes in at least some of the successive images;
C) after the illumination generated by the light source reaches the non-zero amount so as to cause the sufficient reflection, controlling the imaging device to acquire a second number of the successive images, after acquiring the first number of the successive images and while the illumination generated by the light source has at least the non-zero amount to cause the sufficient reflection; and
D) controlling at least the imaging device to acquire at least the first number of the successive images in A) and the second number of the successive images in C) for an amount of time that is sufficiently limited to effectively mitigate significant pupil contraction of the subject's eyes during C).

29. The method of claim 28, wherein C) comprises, after the illumination generated by the light source reaches the non-zero amount to cause the sufficient reflection:
C1) acquiring the second number of the successive images via the imaging device, after the first number of the successive images, while the illumination generated by the light source has at least the non-zero amount to cause the sufficient reflection;
C2) acquiring a third number of the successive images via the imaging device, after the first number of successive images and before the second number of the successive images, while the illumination generated by the light source has at least the non-zero amount to cause the sufficient reflection; and
C3) discarding the third number of the successive images to allow an exposure of the imaging device to stabilize,
wherein the amount of time that the imaging device is operated to acquire at least the first number of the successive images, the second number of the successive images, and the third number of successive images is sufficiently limited to effectively mitigate significant pupil contraction of the subject's eyes during C1).

30. The method of claim 28 wherein, prior to A) and/or during A), the method further comprises:
A-2) controlling at least one audio device to provide at least one audible alert to attract the attention of the subject toward the imaging device.

31. The method of claim 28, wherein the method further comprises:
processing the second number of successive images to analyze a light content in each image of the second number of successive images;
discarding any image of the second number of successive images in which the analyzed light content is excessive or insufficient;
for at least some successive images of the second number of successive images, processing each successive image of the at least some successive images to crop a rectangle containing the subject's eyes to generate a plurality of cropped images; and
processing the plurality of cropped images to select one cropped image of the plurality of cropped images based at least in part on at least one of:
a luminosity of each cropped image of the plurality of cropped images; or
a sharpness of each cropped image of the plurality of cropped images; and
controlling the memory to store the one selected cropped image.

32. A method for facilitating a preliminary diagnosis of an ocular disease in a subject, the method comprising:
A) while the subject is in a sufficiently dark environment that allows the subject's eyes to be effectively dilated, initiating acquisition of successive images of the subject's eyes via an imaging device;
B) during acquisition of a first number of the successive images, increasing an amount of illumination generated by a light source directed at the subject's eyes gradually from zero to a non-zero amount so as to cause sufficient reflection of the illumination generated by the light source from the subject's eyes to the imaging device to effectively render the subject's eyes in at least some of the successive images; and
C) after the illumination generated by the light source reaches the non-zero amount so as to cause the sufficient reflection, acquiring a second number of the successive images via the imaging device, after the first number of the successive images, while the illumination generated by the light source has at least the non-zero amount to cause the sufficient reflection,
wherein an amount of time that the imaging device is operated to acquire at least the first number of the successive images and the second number of the successive images is sufficiently limited to effectively mitigate significant pupil contraction of the subject's eyes during C).

33. The method of claim 32, wherein C) comprises, after the illumination generated by the light source reaches the non-zero amount to cause the sufficient reflection:
C1) acquiring the second number of the successive images via the imaging device, after the first number of the successive images, while the illumination generated by the light source has at least the non-zero amount to cause the sufficient reflection;
C2) acquiring a third number of the successive images via the imaging device, after the first number of the successive images and before the second number of the successive images, while the illumination generated by the light source has at least the non-zero amount to cause the sufficient reflection;
C3) discarding the third number of the successive images to allow an exposure of the imaging device to stabilize; and
C4) storing in a memory the second number of the successive images,
wherein the amount of time that the imaging device is operated to acquire at least the first number of the successive images, the second number of the successive images, and the third number of successive images is sufficiently limited to effectively mitigate significant pupil contraction of the subject's eyes during C1).

34. The method of claim 33, wherein:
in B), the first number of successive images is five;
in C1), the second number of successive images is ten; and
in C2), the third number of successive images is five.
